(19) United States
(12) Patent Application Publication
(10) Pub. No.: US 2010/0215737 A1
(43) Pub. Date: Aug. 26, 2010

(54) COMBINATION PHARMACEUTICAL COMPOSITIONS

(76) Inventor: Ivan Coulter, Dublin (IE)

Correspondence Address: KLARQUIST SPARKMAN, LLP, 121 SW SALMON STREET, SUITE 1600, PORTLAND, OR, 97204, US

(21) Appl. No.: 12/598,386

(22) PCT Filed: May 1, 2008
(86) PCT No.: PCT/IE08/00053

§ 371 Date: Apr. 30, 2010

Related U.S. Application Data
(60) Provisional application No. 60/924,132, filed on May 1, 2007.

(51) Int. Cl. A61K 9/50; A61K 9/64; A61K 31/436; A61K 31/4422; A61K 31/202; A61K 31/165; A61P 25/16; A61P 25/00; A61P 25/28

(52) U.S. Cl. 424/456; 424/490; 514/291; 514/356; 514/560; 514/620

(57) ABSTRACT

A modified release dosage product (5) comprises a plurality of minicapsules or minispheres (1, 2) containing nimodipine, and a plurality of minicapsules or minispheres (3), (4) containing tacrolimus. There are uncoated minicapsules or minispheres (1) encapsulating micronized nimodipine for immediate release and a controlled release polymer coated minicapsule or minisphere (2) encapsulating micronized nimodipine for delayed, sustained, controlled or targeted release. There are uncoated seamless minicapsules (3), the core of which comprises tacrolimus lipid-based formulation for immediate release and a controlled release polymer coated seamless minicapsule (4), the core of which comprises tacrolimus lipid-based formulation for delayed, sustained, controlled release or targeted release. The final dosage form may be a hard gelatin capsule (5).

### INTRODUCTION

Calcium has a pervasive role in regulating brain function, for example plasticity, glucose metabolism, cerebrovascular regulation, neurotransmitter synthesis and release, axonal transport and neuronal dendritic claw formation. Calcium ions are ubiquitous messengers linking membrane excitation to subsequent intracellular molecular responses. Changes in calcium homeostasis are an aspect of aging that may have implications for higher cerebral functions. Therefore, pharmaceutical or other interventions that reduce negative changes or maintain healthy calcium homeostasis have the potential to improve brain health and thus prevent disease or provide treatments for various neurological and neurodegenerative diseases.

Nimodipine, a member of the dihydropyrimidine class of drugs, belongs to the class of pharmacological agents known as calcium channel blockers. The contractile processes of smooth muscle cells are dependent upon calcium ions, which enter these cells during depolarisation as slow ionic transmembrane currents. Nimodipine inhibits calcium ion transfer into these cells and thus inhibits contractions of vascular smooth muscle. Nimodipine is a yellow crystalline substance, practically insoluble in water. Nimodipine is typically formulated as soft gelatin capsule for oral administration.

Nimodipine is indicated for the improvement of neurological outcome by reducing the incidence and severity of ischemic deficits in patients with subarachnoid hemorrhage from ruptured intracranial berry aneurysms regardless of their post-ictus neurological condition. The precise mode of action is not clear. In patients with Hunt and Hess Grades I-III, nimodipine significantly reduces the risk of cerebral infarction and poor outcome in (subarachnoid hemorrhage) SAH. In patients with Hunt and Hess Grades IV and V, nimodipine improves recovery while decreasing severe disability and vegetative survival in SAH patients with poor neurological status.

In addition to the current subarachnoid hemorrhage indication, as an highly lipophilic calcium channel blocker that can pass the blood brain barrier and enter the cerebral vasculature, nimodipine, alone or in combination with other therapeutically active entities, may have a number of other activities in the brain, including cognitive enhancement, reducing neuropathic pain, alleviating stroke ailments, treating or preventing cluster headaches or migraines and preventing or treating neurodegenerative conditions, including Parkinson's disease and Alzheimer's disease. Additionally, in combination with morphine nimodipine has been shown both to not only reduce the concentration of morphine required to reduce pain, but also extend the duration of pain reduction. Despite the potential applications, none of the above potential indications is attractive if the drug requires to be administered up to six times a day and has a potentially fatal capacity to induce hypotension.

Tacrolimus, a macrolide immunosuppressant, is available in both oral and iv formulations, it is indicated for the prophylaxis of organ rejection in patients receiving allogenic liver, kidney or heart transplants. Branded as Prograf®, it is also approved in Japan for patients undergoing bone marrow transplant, and for myasthenia gravis and rheumatoid arthritis.

Evidence suggests that that calcineurin regulation or dysregulation plays a role in brain damage and thus pharmacological intervention has the potential to limit the short- and long-term effects of calcineurin malfunction. The proposed role of calcineurin in the neuroprotective mechanism could involve a number of cellular processes and may involve the interaction of certain complexes with components associated with calcium channel blockers. Additionally, calcineurin activity may be associated with apoptosis leading to ischemic brain damage with the hypothesis that inhibiting calcineurin activity reduces apoptotic death and therefore reduces ischemic insult. In addition to preventing damage, calcineurin inhibitors have demonstrated a capacity to enhance neuronal dendritic claw formation, with the implied suggestion that this may be of particular benefit to prevent the progression of neurodegenerative diseases such as, but not limited to Parkinson's disease and Alzheimer's disease.

Based on the known role of calcium homeostasis and activity in the brain, calcium channel blockers and calcineurin inhibitors have significant potential to prevent or treat a number of brain or neurological conditions. Indeed, the calcium channel blocker nimodipine has demonstrated effectiveness in a range of conditions, primarily subarachnoid hemorrhage, while the calcineurin inhibitors cyclosporin A and tacrolimus have demonstrated effectiveness in various ischemic-based physiological or trauma-induced conditions.

Logically, for a pharmaceutical agent to have activity on its intended target receptor in the body, the agent must reach the receptor intact, in free solution and in sufficient concentrations to exert its activity. Following oral administration, a drug intended for the brain must first overcome the gastrointestinal barrier, intestinal and hepatic metabolism before crossing the blood-brain-barrier (BBB). The intestinal and hepatic barriers are metabolic, the principal enzyme family that breaks down various drugs is cytochrome P450 and if the drug is a substrate for this enzyme the amount reaching the bloodstream can vary extensively. The BBB evolved to prevent the passage of toxins into the brain. While it permits certain entities, including lipophilic agents, to pass through into the brain, the presence of the P-glycoprotein (PgP) efflux pump, whose role it is to eject perceived exogenous toxins from cells, causes brain concentrations of many drugs to be very low and often very variable. It is noteworthy that many drugs that are substrates for either cytochrome P450 or PgP also inhibit or reduce the activity or, indeed, saturate the enzyme and resulting in increased bioavailability of susceptible drugs. The other important aspects to consider are the solubility and permeability of the drug, if the drug is not in a soluble form it will not interact with its intended receptor efficiently and if it is not permeable it will not pass from the intestinal lumen into the bloodstream nor pass from the bloodstream into the brain, via the BBB. Thus, modulating solubility and permeability can increase or regulate the concentration of drug absorbed into the body from the intestine or the consequent passage into the brain.

Many calcium channel and calcineurin inhibitors are poorly soluble and are substrates for both the cytochrome P450 and PgP enzymes and efflux pumps, leading to variable bioavailability. Also, many such drugs also inhibit or regulate the activity of cytochrome P450 and PgP enzymes and efflux pumps and thus may regulate the transport of drugs from the intestine into the bloodstream and from the bloodstream into the brain. Regarding solubility and permeability, many, mostly lipid-based, formulations improve the solubility and often also the permeability of poorly soluble drugs. The present invention enables the solubility and permeability enhancement of drugs using various formulation technology approaches. The resulting solutions permit not only controlled release of such formulations, but also permit the development of novel combinations of nimodipine and tacrolimus with the intention that the combination will act not only in a pharmacologically synergistic manner in the brain, but also modulate cytochrome P450 enzyme and PgP efflux pump activity such that more or one or both drug first enters the bloodstream in a less variable manner and thereafter more or one or both permeates the BBB and enters the brain in a less variable manner. Overall, the development of novel and improved combination therapies will result in improved disease management and outcome.

The current invention enables the development of combination therapies comprising a calcium channel blocker and a calcineurin inhibitor, both sustained released and which act complementarily to modulate the cytochrome P450 enzyme and PgP efflux pump activity to ensure enhanced and less variable bioavailability in the bloodstream and brain and to act in pharmacological complementary and synergy within the brain. As such, the invention will provide benefit in a range of neurological and traumatic CNS conditions.

### STATEMENTS OF INVENTION

According to the invention there is provided a modified release dosage product comprising:—
a plurality of minicapsules or minispheres containing nimodipine; and
a plurality of minicapsules or minispheres containing tacrolimus.

In one embodiment, when exposed to a use environment substantially all of the nimodipine and substantially all of the tacrolimus are released within a 24 hour period.

In one case the minicapsules or minispheres containing nimodipine comprise a first population containing nimodipine for immediate release and a second population containing nimodipine for controlled release. The first population may comprise minispheres containing nimodipine in a solid form for immediate release. The second population may comprise minicapsules containing nimodipine, the capsule having a controlled release coating. The second population may comprise a first sub-population for release of nimodipine over a period of from 0 to 12 hours and a second sub-population for release of nimodipine over a period of from 12 to 24 hours.

In one embodiment the minicapsules or minispheres containing tacrolimus comprise a first population containing tacrolimus for immediate release and a second population containing tacrolimus for controlled release. In one case the first population comprises tacrolimus in a liquid form encapsulated within minicapsules. In one embodiment the second population comprises minicapsules containing tacrolimus, the capsule having a controlled release coating. The second population may comprise sub-population for release of tacrolimus over a period of from 0 to 24 hours.

In one embodiment, when exposed to a use environment, more than 40% of the nimodipine and more than 40% of the tacrolimus are released within 12 hours. In one case when exposed to a use environment, less than 15% of the tacrolimus and less than 15% of the nimodipine are released within 1 hour. In one case when exposed to a use environment, less than 30% of the nimodipine and less than 30% of the tacrolimus are released within 4 hours.

In one embodiment the modified release dosage product comprises a hard gelatin capsule containing the nimodipine minicapsules or minispheres and the tacrolimus minicapsules or minispheres.

The modified release dosage product may comprise a sachet containing the nimodipine minicapsules or minispheres and the tacrolimus minicapsules or minispheres.

Alternatively the modified release dosage product comprises a pellet containing the nimodipine minicapsules or minispheres and the tacrolimus minicapsules or minispheres.

The modified release dosage product may comprise a naso-gastric feeding product containing the nimodipine minicapsules or minispheres and the tacrolimus minicapsules or minispheres.

The invention also provides a modified release dosage product comprising:—
a plurality of minicapsules or minispheres containing a calcium channel blocker such as nimodipine; and/or
a plurality of minicapsules or minispheres containing a calcineurin inhibitor such as tacrolimus.

The product may be used for the treatment/prevention of subarachnoid hemorrhage; for the treatment/prevention of stroke; for the treatment/prevention of transient cerebral ischemia; for the treatment/prevention of focal cerebral ischemia; for the treatment/prevention of Parkinson's disease; for the treatment/prevention of Restless Leg Syndrome; for the treatment/prevention of Alzheimer's disease; for the treatment/prevention of ALS; and/or for the treatment/prevention of vascular dementia.

In one embodiment the product contains high purity eicosapentaenoic acid (EPA).

In another embodiment the product contains high purity docosahexaenoic acid (DHA).

In one case the product is used for the treatment/prevention of Huntington's disease.

The modified release dosage product may contain AChEI, such as, Huperzine.

The modified release dosage product may contain safinamide. In this case the product may be used for Parkinson's disease or Restless Leg Syndrome.

In another embodiment the modified release dosage product contains a dopamine analogue or agonist such as levodopa, cabergoline, bromocriptine, apomorphine, pergolide mesylate, pramipexole or ropinirole. In this case the product is used for Parkinson's disease or Restless Leg Syndrome.

In one aspect the invention provides a modified release dosage product wherein the minicapsule core contains tacrolimus in a liquid, lipid-based formulation and the encapsulating material contains micronized nimodipine.

In one embodiment the modified release dosage product comprises a plurality of minicapsules or minispheres containing a hydroxylase inhibitor such as hydralazine. The product may combine with a nitric oxide donor such as nitroglycerine.

In another embodiment the modified release dosage product comprises a plurality of minicapsules or minispheres containing an anti-coagulant. The anti-coagulant may be selected from any one or more of aspirin, clopidogral or ticlopidine.

In a further embodiment the modified release dosage product comprises a plurality of minicapsules or minispheres containing an angiotensin II receptor antagonist such as losartan.

In a further embodiment the modified release dosage product comprises a plurality of minicapsules or minispheres containing a nootrophic such as piracetem.

In another embodiment the modified release dosage product comprises a plurality of minicapsules or minispheres containing a NMDA receptor antagonist such as memantine hydrochloride.

In a still further embodiment the modified release dosage product comprises a plurality of minicapsules or minispheres containing a xanthine, such as propentofylline or theophylline.

In yet another embodiment the modified release dosage product comprises a plurality of minicapsules or minispheres containing a cholinesterase inhibitor. The cholinesterase inhibitor may be any one of huperzine A, tacrine, donepezil, galanthamine or rivastigmine.

In a further embodiment the modified release dosage product comprises a plurality of minicapsules or minispheres containing an opiate. The opiate may be any one of morphine, morphine sulphate, oxycodone, hydrocodone, fentanyl or tramadol.

In another embodiment the modified release dosage product comprises a plurality of minicapsules or minispheres containing a migraine or cluster headache treatment or prophylactic. The migraine treatment or prophylactic may be any one or combination of aspirin, paracetamol, naproxen or NO-donor-conjugated naproxen, ibuprofen or NO-donor conjugated ibuprofen, sumatriptan or zolmitriptan.

In another embodiment the modified release dosage product comprises a plurality of minicapsules or minispheres containing a depression treatment or prophylactic. The migraine treatment or prophylactic is any one any one or combination of lithium, valproate, olanzapine, carbamazapine, lamotrigine or eicospentaenoic acid (EPA) and docosahexaenoic acid (DHA) omega-3 fatty acids.

In one aspect the modified dosage product comprises at least one minicapsule population filled into hard gelatin capsules.

In another aspect the product comprises at least one minicapsule population filled into a sachet.

In a further aspect the product comprises at least one minicapsule population contained within a wide gauge syringe or a unit that is compatible with tube delivery.

The product may comprise at least one minicapsule population in the form of a sprinkle.

At least one minicapsule population may be suspended in oil as a lubricant.

In one aspect the product comprises at least one minicapsule population formulated as a suppository for rectal or vaginal administration.

In another aspect the product comprises at least one minicapsule population formulated for buccal delivery. The product may comprise at least one minicapsule population contained in a bioadhesive polymer strip.

The product may comprise at least one minicapsule population formulated for sublingual delivery. At least one minicapsule population may be contained in a bioadhesive polymer strip.

In another aspect the product comprises at least one minicapsule population contained in a sprinkle form.

According to one aspect the invention provides modified release solid dosage product comprising nimodipine, wherein when exposed to a use environment more than 40% of the nimodipine is released within 12 hours and wherein the Tₘₐₓ is reached within 6 hours. In one embodiment substantially all of any remaining nimodipine is released between 12 and 24 hours. The product may comprise solid minicapsules containing nimodipine. The product may comprise one or more populations of minicapsules, at least one of which population comprising minicapsules which are coated with a release agent. The product is suitable for once daily administration. In one case the plasma concentration remains within 7.5 ng/ml and 15 ng/ml for 75% of the time in a 24 hour period. The modified release dosage product may comprise from 90 mg to 450 mg of nimodipine.

In one case micronized nimodipine is present in the minicapsule in an amount of from 10 to 70% w/w, preferably in an amount of from 30 to 45% w/w.

According to another aspect of the invention there is provided an oral tacrolimus composition comprising minicapsules having a core containing tacrolimus in a solubilised liquid form.

In one embodiment the minicapsules have a release profile to release pre-solubilised tacrolimus in the small intestine.

In one embodiment the minicapsules have a release profile to release pre-solubilised tacrolimus in the ileum.

In one embodiment the minicapsules have a release profile to release pre-solubilised tacrolimus in the colon.

In one case tacrolimus is present in the core in an amount of from 0.5 to 25% w/w, preferably in an amount of from 2.5 to 15% w/w.

In one embodiment when exposed to a use environment less than 30% of the tacrolimus is released within 1 hour, preferably when exposed to a use environment less than 20% of the tacrolimus is released within 1 hour.

In one embodiment when exposed to a use environment less than 60% of the tacrolimus is released within 4 hours, preferably when exposed to a use environment less than 35% of the tacrolimus is released within 4 hours.

In one embodiment when exposed to a use environment less than 90% of the tacrolimus is released within 12 hours, preferably when exposed to a use environment less than 65% of the tacrolimus is released within 12 hours.

In one case when exposed to a use environment less than or equal to 100% of the tacrolimus is released within 24 hours.

In one embodiment when exposed to a use environment less than 20% of the tacrolimus is released within 1 hour, less than 35% of the tacrolimus is released within 4 hours, less than 65% of the tacrolimus is released within 12 hours, and substantially all of the remaining tacrolimus is released between 12 and 24 hours.

The minicapsules may comprise a solid shell containing the solubilised tacrolimus. The minicapsules may be modified to provide the release profile. A modified release may be attributable to a polymer coating. The polymeric material may, for example, be a methacrylate, or ethylcellulose. The polymeric material may be a composite of methacrylate and ethylcellulose.

In one embodiment the coating includes a dissolution enhancing agent. The dissolution enhancing agent may be degraded by bacteria normally present in the gastrointestinal tract. The dissolution enhancing agent may be selected from one or more of, pectin, amylose and alginate. The dissolution enhancing agent can be present in an amount of from 0.5 to 2% w/w of ethylcellulose.

In one embodiment the core comprises tacrolimus, a solubilisation agent, a co-emulsifier, a surfactant, a permeability enhancer and a carrier. The solubilisation agent may comprise ethanol. The solubilisation agent may comprise triglycerides. The co-emulsifying agent may comprise fatty acid ester complexes. The surfactant agent may comprise fatty acid ester complexes. The permeability enhancing agent may comprise fatty acid ester complexes. The carrier may comprise a hydrophobic liquid. The hydrophobic liquid may comprise an oil such as olive oil.

In one embodiment the composition comprises a first population of minicapsules comprising tacrolimus and a second population of minicapsules comprising tacrolimus. The first population may comprise uncoated minicapsules. The second population may comprise coated minicapsules.

In one embodiment the composition comprises from 10 to 40% w/w uncoated minicapsules and from 60 to 90% w/w coated minicapsules.

In one case there are about 25% w/w of uncoated minicapsules and about 75% w/w of coated minicapsules.

In one embodiment tacrolimus is released along the gastrointestinal tract in a form that maximises systemic absorption.

In another embodiment there is a combination of controlled release micronized nimodipine and tacrolimus with release profiles as described heretofore, said combination being comprised of solid uncoated and coated minispheres as well as uncoated and coated liquid-filled minicapsules.

In another embodiment the liquid filled core contains solubilised tacrolimus while the encapsulating shell contains micronized nimodipine. The resulting liquid-filled minicapsule may then remain uncoated or be coated with a controlled release polymer.

In one embodiment the gelling or encapsulating agent is gelatin, animal or non-animal derived.

In another embodiment the gelling or encapsulating agent is a non-gelatin entity, including, but not limited to, alginate, pectin, carrageenan or the like. In one case the active pharmaceutical ingredient is an NO-donor conjugated Nimodipine.

In one embodiment either single product or the combined products is used to treat or prevent subarachnoid haemorrhage.

In another embodiment either single product or the combined products is used to treat or prevent stroke or transient ischemia. Additionally, either product or the combined products may be combined with a hydroxylase inhibitor, released concurrent or sequentially with either nimdipine or tacrolimus or both. The hydroxylase inhibitor may be hydralazine. In another case the product is combined with a nitric oxide donor such as nitroglycerine. In a further case the product is combined with an anti-coagulant which may be selected from any one or more of aspirin, clopidogral or ticlopidine. In another case the product is combined with an angiotensin II receptor antagonist such as losartan. Additionally, NO-donor conjugated hydralazine or any of the above may be included.

In another embodiment either single product or the combined products is used to treat or prevent Alzheimer's disease and other dementias. In this case the product may be combined with a nootrophic. The nootrophic may be piracetem. In another case either single product or the combined products is combined with a NMDA receptor antagonist such as memantine hydrochloride. In a further case either single product or the combined products is combined with a xanthine such as propentofylline. In another case either single product or the combined products is combined with a cholinesterase inhibitor. The cholinesterase inhibitor may be any one of huperzine A, tacrine, donepezil, galanthamine or rivastigmine.

In a further embodiment either single product or the combined products is used to treat or prevent neurodegenerative disease. The neurodegenerative disease may be Parkinson's disease or Restless Leg Syndrome. In this case either single product or the combined products may be combined with safinamide. In a further case the product is combined with a dopamine analogue or agonist. The dopamine analogue or agonist may be any one of levodopa, cabergoline, bromocriptine, apomorphine, pergolide mesylate, pramipexole or ropinirole hydrochloric.

In another embodiment the product is used to treat or prevent Meniere's disease. The product may be used to treat or prevent vertigo.

In another embodiment the product, nimodipine alone or in combination with tacrolimus, is used to treat or prevent neuropathic pain. In this case the product may be combined with an opiate. The opiate may be any one of morphine, morphine sulphate, oxycodone, hydrocodone, fentanyl or tramadol. In another case the product is combined with pregabalin. In a further case the product is combined with an α-aminoamide. In another case the product may be combined with naproxen or an NO-donor conjugated naproxen.

In one embodiment the product is a single-layer minicapsule containing Nimodipine or a NO-donor conjugate thereof and one or more other active pharmaceutical ingredient.

In another embodiment the product is a two-layer minicapsule. The core and shell may contain the same active pharmaceutical ingredient. Alternatively the core contains tacrolimus and the shell contains micronized nimodipine. In one case the core formulation is controlled release and the shell is immediate release. In another case the core formulation is controlled release and the shell is controlled release.

In another embodiment the product, either two-layer minicapsule or single layer solid minisphere may additionally contain plant, animal, dairy, algae or marine extracts, said extracts having formulation enhancing or health promoting properties.

In one embodiment the two-layer minicapsule is coated with a controlled release polymer or materials.

In one aspect the product comprises at least one minicapsule population filled into hard gelatin capsules.

In another aspect the product comprises at least one minicapsule population filled into a sachet.

The product may comprise at least one minicapsule population contained within a wide gauge syringe or a unit that is compatible with tube delivery.

In another aspect the product comprises at least one minicapsule population in the form of a sprinkle.

At least one minicapsule population may be suspended in oil as a lubricant.

In one case the product comprises at least one minicapsule population formulated as a suppository for rectal or vaginal administration.

The product may comprise at least one minicapsule population formulated for buccal delivery.

The product may comprise at least one minicapsule population contained in a bioadhesive polymer strip.

In another case the product comprises at least one minicapsule population formulated for sublingual delivery.

At least one minicapsule population may be contained in a bioadhesive polymer strip.

In a further case the product comprises at least one minicapsule population contained in a sprinkle form.

The minicapsules may contain a disintegrant.

The minicapsules may contain a muco-adhesive or bio-adhesive.

The minicapsules may contain a permeability enhancer.

The minicapsules may contain a taste-masking agent.

In one embodiment the product comprises minispheres.

### BRIEF DESCRIPTION OF THE DRAWINGS

The invention will be more clearly understood from the following description of an embodiment thereof, given by way of example only, with reference to the accompanying drawings, in which:—

FIG. 1 illustrates the dissolution profile of an average of two batches from nimodipine solid minispheres over a 24 hour period. The profile represents release of 30 mg nimodipine from a blend of three distinct populations of minisphere: 5 mg uncoated, 6 mg coated with 15% weight gain Surelease® and 19 mg coated with 30% weight gain Surelease®. This product profile is suited to once-daily administration of nimodipine;

FIG. 2 illustrates the dissolution profile of an average of two batches from nimodipine solid minispheres over a 24 hour period. The profile represents release of 30 mg nimodipine from a blend of two distinct populations of minisphere: 9 mg uncoated and 21 mg coated with 20% weight gain Surelease®. This product profile is suited to twice daily administration of nimodipine;

FIG. 3 illustrates the dissolution profile of an average of two batches from nimodipine solid minispheres over a 24 hour period. The profile represents release of 30 mg nimodipine from a blend of two distinct populations of minisphere: 9 mg uncoated and 21 mg coated with 15% weight gain Surelease®. This product profile is suited to twice daily administration of nimodipine;

FIG. 4 illustrates the dissolution profile of an average of six batches from nimodipine solid minispheres over a 24 hour period. The profile represents release of 180 mg nimodipine from a blend of three distinct populations of minisphere: 14.9 mg uncoated, 35.6 mg coated with 7.5% weight gain Surelease® and 130.5 mg coated with 30% weight gain Surelease®. This product profile is suited to once-daily administration of nimodipine;

FIG. 5 illustrates the dissolution profile from an average of two batches of 30 mg 3-layer nimodipine uncoated minicapsules. The profile demonstrates that the core formulation is inherently sustained release;

FIG. 6 illustrates the dissolution profile from an average of two batches of 30 mg 3-layer nimodipine minicapsules over 24 hours. The 3-layer minicapsules were coated with a 6.5% weight gain blend of Eudragit® RS and Eudragit® RL to provide external controlled release as well as the inherent internal sustained release inherent to such 3-layer minicapsules, as demonstrated in FIG. 4;

FIG. 7 illustrates the dissolution profile from an average of two batches of 30 mg 3-layer nimodipine minicapsules over 24 hours. The 3-layer minicapsules were coated with a 13.5% weight gain blend of Eudragit® RS and Eudragit® RL to provide external controlled release as well as the inherent internal sustained release inherent to such 3-layer minicapsules, as demonstrated in FIG. 4.

FIG. 8 illustrates the pharmacokinetic plasma profile for the test product (180 mg Nimodipine as per FIG. 7) versus 6×30 mg Nimotop™ over a 24 hour period. The pharmacokinetic study represents the average of 20 healthy male volunteers and the plasma concentration is measured in ng/ml. This product profile is suited to once- or twice-daily administration.

FIG. 9 is a graph showing the dissolution profile for uncoated tacrolimus minicapsules;

FIG. 10 is a graph showing the dissolution profile for tacrolimus minicapsules coated with 12.5% Eudragit™ RS30D followed by 25% Eudragit™ FS30D;

FIG. 11 is a graph showing the dissolution profile for composite tacrolimus minicapsules—30% uncoated (immediate release) and 70% coated with 12.5% Eudragit™ RS30D followed by 25% Eudragit™ FS30D;

FIG. 12 is a graph showing the dissolution profile for 15% weight gain Eudragit™ profile for 15% weight gain Eudragit™ RS30D-coated tacrolimus minicapsules;

FIG. 13 is a graph showing the dissolution profile for 15% weight gain Eudragit™RS30D/25% weight gain Surlease®-coated tacrolimus minicapsules;

FIG. 14 is a graph showing the dissolution profile for 15% weight gain variable RS/RL coatings;

FIG. 15 illustrates the dissolution profile of an average of three batches from solid, hydralazine-containing lipid-gelatin-based minispheres with a 20% weight gain Eudragit™ RS30D sustained release polymer coating. The product is suited to once-daily administration of hydralazine;

FIG. 16 illustrates the dissolution profile of an average of three batches from solid, hydralazine-containing lipid-gelatin-based minispheres with a 30% weight gain Eudragit™ RS30D sustained release polymer coating. The product is suited to a delayed release administration form of hydralazine for chronotherapy or sequential therapy combinations;

FIG. 17 is a schematic illustration of a liquid-filled minicapsules and solid minispheres of the type used in the formulations of the invention. 1 represents an uncoated solid, gelatine-based minicapsule or minisphere encapsulating micronized active (such as nimodipine) for immediate release. 2 represents a controlled release polymer coated solid, gelatine-based minicapsule or minisphere encapsulating micronized active (such as nimodipine) for delayed, sustained, controlled or targeted release. 3 represents an uncoated solid minicapsule, the core of which comprises an active (such as tacrolimus) lipid-based liquid formulation encapsulated in a solid gelatin shell for immediate release. 4 represents a controlled release polymer coated solid gelatin shell encapsulated lipid-based liquid formulation containing an active (such as tacrolimus) for delayed, sustained, controlled release or targeted release. 5 represents a final dosage form, namely a hard gelatin capsule containing any combination of 1, 2, 3, or 4.

### DETAILED DESCRIPTION

### Role of Calcium in Neural Maintenance

Sustained calcium (Ca²⁺) influx through glutamate receptor channels is thought to represent a final common pathway of neuronal cell death that is associated with a number of neurodegenerative diseases such as epilepsy, hypoxia-ischemia, hypoglycemia, Alzheimer Disease, and schizophrenia (Trends Neurosci. 18, 58-60 (1995)). Although large increases in [Ca²⁺]ᵢ result in acute and delayed cell death (Yu et al., 2001), recent evidence from mammalian neurons suggests that moderate increases of [Ca²⁺]ᵢ (50-200 nmol l⁻¹) play a neuroprotective role in hypoxia and glucose deprivation (Bickler and Fahlman, 2004). Therefore, regulation of calcium concentration in brain cells is considered to be essential to the maintenance of healthy brain function.

A multitude of drug classes has been developed to modulate and control calcium levels to enable the management of a number of diseases, including cardiovascular, neuropathic pain and neurological diseases. Two of the main classes of drug developed to control calcium levels are calcium channel blockers and calcineurin inhibitors. To enter the brain, to ensure that passage through the blood brain barrier is possible, such drugs benefit from being lipophilic in nature. A drawback of many such drugs is poor solubility and permeability, the former is attributed mainly to the physicochemical properties, the latter to physiological mechanisms that have evolved to protect the body from exotoxins. The principal physiological mechanisms are associated with a family of metabolic enzymes known as the Cytochrome P450 (CYP) family and an efflux pump protein known as P-glycoprotein (PgP).

Drug delivery mechanisms have been developed to address solubility and permeability and the current invention uses innovative approaches to bundle a number of drug delivery approaches to facilitate the controlled release of calcium channel blockers and calcineurin inhibitors, either individually or collectively. Apart form pharmacological synergies in the brain, the co-administration of calcium channel blockers and calcineurin inhibitors may also regulate CYP and PgP to ensure greater plasma and cerebrospinal fluid concentrations while reducing the variability of such concentrations.

### Nimodipine

Nimodipine, a member of the dihydropyrimidine class of drugs, belongs to the class of pharmacological agents known as calcium channel blockers. The contractile processes of smooth muscle cells are dependent upon calcium ions, which enter these cells during depolarisation as slow ionic transmembrane currents. Nimodipine inhibits calcium ion transfer into these cells and thus inhibits contractions of vascular smooth muscle. Nimodipine is a yellow crystalline substance, practically insoluble in water. Nimodipine is typically formulated as soft gelatin capsule for oral administration.

### Nimodipine Bioavailability

Currently, due to limited solubility, Nimodipine is available only as a soft-gel capsule, each capsule containing a 30 mg dose. As nimodipine is a substrate for cytochrome P450 3A4 isoenzyme and the efflux pump P-glycoprotein (PgP), it is therefore extensively and presystemically metabolized or expelled from cells, resulting in a relative bioavailability of approximately 18%. Thus, a relatively high dose and frequency regime is required. Due to limited stability, one or two 30 mg large-soft gel capsules are administered up to six times per day, which results in spikes of high plasma and cerebrospinal fluid (CSF) concentration with potential serious side-effects and is also a major inconvenience that leads to poor compliance.

A further difficulty is that, many patients who present with subarachnoid hemorrhage are variously incapacitated and thus require feeding through naso-gastric tubes. As such patients are unable to swallow carers must syringe the contents of the soft-gel capsules out and to feed the drug solution through the feeding tube, a process that must be repeated up to six times per day.

Apart from the inconvenience caused to patient and carer, the resulting high dose nimodipine acts in a bolus-like manner whereby the plasma concentration spikes, often leading to hypotension. Also, the extreme peak to trough swing that may result in a reflex increase in systolic flow velocities (PSV) or cerebral vasospasms, events that are prognostic of poor patient outcome.

### Tacrolimus

Tacrolimus, a macrolide agent, inhibits T-lymphocyte activation through a process that is thought to involve it binding to an intracellular protein, FKBP-12. A hydrophobic complex of tacrolimus-FKBP-12, calcium, calmodulin, and calcineurin is then formed and the phosphatase activity of calcineurin inhibited. This effect may prevent the dephosphorylation and translocation of nuclear factor of activated T-cells (NF-AT), a nuclear component thought to initiate gene transcription for the formation of lymphokines (such as interleukin-2, gamma interferon). The resulting inhibition of T-lymphocyte activation leads to potent immunosuppression (Prograf® Patient Information Brochure (Astellas)). In addition to its indicated role in a range of solid organ transplant anti-rejection uses, various studies indicate clearly that the tacrolimus neurotrophic and neuroregenerative effects have potential efficacy in a range of neurological conditions, including, but not limited to stroke, transient and permanent focal ischemia as well as transient global ischemia. By implication, based on the known mode of action, tacrolimus has potential to treat or prevent a number of other neurological conditions, including, but not limited to, Parkinson's disease, Restless Leg Syndrome, Alzheimer's disease and so forth.

### Tacrolimus Bioavailability

Tacrolimus is differentially absorbed from different regions of the gastrointestinal tract, being optimally absorbed from the small intestine, with ileum and colonic absorption efficiency dropping to half that observed for the small intestine. Also, a food effect is observed. After absorption from the gastrointestinal tract, drug effects persist for 8-12 hours after oral administration of conventional IR tablets. The total dosage is typically in the range of 2.5-10 mg per day, in exceptional cases rising to 20 mg/day. Under conventional dosage regimes, Tacrolimus is given twice daily, typically with one dose given before breakfast and a second dose given in the late afternoon. Adverse effects, due to the initial rapid absorption from the small intestine results in above therapeutic plasma concentrations, associated with tacrolimus treatment include nephrotoxicity, neurotoxicity and the development of patient infection due to immunosuppression.

As with other calcineurin inhibitors such as cyclosporine A, Tacrolimus has a narrow therapeutic index. Chronic tacrolimus blood concentrations above the therapeutic target concentration of 15 ng/ml increases the risk of tacrolimus-related toxicity, the principal adverse effects include kidney and liver damage as well as an increased incidence of diabetes, the latter affecting 20% of all patients. It is suggested that, in promoting long term organ graft survival, reducing immunosuppressant-induced nephrotoxicity may be as important as reducing the incidence and occurrence of acute organ rejection episodes. The marketed product, Prograf®, is rapidly absorbed resulting in a spike in the blood concentration above long term considered safe concentration of 15 ng/ml. Furthermore, the elimination of product results in a sub-therapeutic blood concentration of less than 5 ng/ml after about 8 hours. The fact that the drug must be administered twice daily exposes patients to twice-daily toxic concentration as well as periods of sub-therapeutic doses. Additionally, twice-daily dosing is an inconvenient regimen for patients. Therefore, for improved safety, better disease management and patient-convenience, a once-daily, low dose Tacrolimus formulation is highly desirable.

A once-daily formulation of tacrolimus is known. The formulation process consists of tacrolimus being granulated with dehydrated ethanol, ethylcellulose, hypromellose and lactose monohydrate. The hypromellose system modifies the drug release profile by forming a polymer gel layer and the ethylcellulose diffusion matrix system modifies the release profile by controlling water penetration and thus drug release. The resulting paste undergoes drying and sizing to produce intermediate granules. The granules are then mixed with lactose monohydrate and magnesium stearate and that mixture is filled into capsules. The formulation results in dissolution of 90% drug release at 6 to 12 hours. One potential problem with the above once-daily product results in an initial spike in the drug plasma concentration, with the potential to cause unwanted side effects. Therefore, a once-daily, steady-state product with a safer patient profile is desirable.

### Role of Cytochrome P450 and P-Glycoprotein on Drug Bioavailability

It is well documented that the bioavailability of both nimodipine and tacrolimus is adversely affected by Cytochrome P450 metabolism and P-glycoprotein efflux. The result of metabolism and efflux is variable inter- and intra-subject variability of plasma and cerebrospinal fluids concentrations. The cytochrome P450 acts at the intestinal wall and in the liver while the PgP works in the membrane of many cells, primarily those of the intestinal villi and the blood brain barrier.

Intestinal metabolism and active extrusion of absorbed drug have recently been recognised as major determinants of oral bioavailability. Cytochrome CYP (CYP) 3A, the major phase I drug metabolising enzyme in humans, and the multidrug efflux pump, P-glycoprotein, are present at high levels in the villus tip of enterocytes in the gastrointestinal tract, the primary site of absorption for orally administered drugs. The importance of CYP and P-glycoprotein in limiting oral drug delivery is suggested to us by their joint presence in small intestinal enterocytes, by the significant overlap in their substrate specificities, and by the poor oral bioavailability of joint substrates for these 2 proteins. These proteins are induced or inhibited by many of the same compounds. A growing number of preclinical and clinical studies have demonstrated that the oral bioavailability of many CYP and/or P-glycoprotein substrate drugs can be increased by concomitant administration of CYP inhibitors and/or P-glycoprotein inhibitors (Clinical Pharmacokinetics. 40(3):159-168, 2001). Due to the co-localised distribution of CYP and PgP proteins along the gastrointestinal tract, albeit in differing concentrations, the co-release of drugs that are substrates or inhibitors, such as nimodipine and tacrolimus, of either protein along the gastrointestinal tract may serve to increase the absorption of one, both or more drug from the intestinal lumen into the bloodstream and to reduce the variability of absorption.

Following oral administration, the small intestine is the initial site of metabolism of ingested xenobiotics, including therapeutic drugs, through reactions catalyzed primarily by the cytochrome P450 (CYP) oxidative xenobiotic-metabolizing enzyme system. The CYPs are the principle enzymes involved in the biotransformation of drugs and other foreign compounds. They comprise a superfamily of hemeproteins that contain a single-iron protoporphyrin IX prosthetic group. This superfamily is subdivided into families and subfamilies that are classified solely on the basis of amino acid sequence homology. At least 14 CYP gene families have been identified in mammals (Nelson et al., 1996). However, only three main CYP gene families, CYP1, CYP2, and CYP3 currently are thought to be responsible for drug metabolism.

Unlike the liver in which the distribution of CYP enzymes is relatively homogeneous (Debri et al., 1995), the distribution of these enzymes is not uniform along the length of the small intestine nor along the villi within a cross-section of mucosa. Both the content and activity of cytochrome CYP was higher in the proximal than that in the distal small intestine (Peters and Kremers, 1989). The average total cytochrome CYP content in human intestine, about 20 pmol/mg microsomal protein, was found to be much lower than that in the liver (300 pmol/mg microsomal protein) (Peters and Kremers, 1989; Shimada et al., 1994) and it has been shown that CYP expression varies along the length of the small intestine. Median values of 31, 23, and 17 pmol/mg microsomal protein were measured in human duodenum, distal jejunum, and distal ileum, respectively (Thummel et al., 1997). CYP is a superfamily of heme-containing monooxygenases (Nelson et al., 2004), many of which are expressed in the small intestine (Kaminsky and Fasco, 1992; Kaminsky and Zhang, 2003), and are active in the bioactivation or detoxification of numerous toxic chemicals, carcinogens, and therapeutic drugs. It has been proposed that the expression levels and the activities of CYP enzymes in the small intestine directly affect the bioavailability of many drugs (Suzuki and Sugiyama, 2000; Doherty and Charman, 2002; Ding and Kaminsky, 2003). The expression of small-intestinal CYP enzymes is regulated by exposure to dietary and xenobiotic compounds, (Kaminsky and Fasco, 1992; Zhang et al., 1996; Zhang et al., 2003), as well as by pathologic conditions such as inflammation (Kalitsky-Szirtes et al., 2004; Xu et al., 2006). Also, there is significant inter- and intra-subject variability in the expression of CYP genes amongst individuals, leading to widely variable absorption of drugs that are substrates of CYP> Thus, compounds that inhibit CYP may enhance and improve the regulation of drugs that have been administered oral leading to a more consistent plasma drug concentration and better disease management.

PgP is found in the luminal plasma membrane of brain capillary endothelial cells (BCEC) and have been shown to pump drugs such as cyclosporine A, a member of the calcineurin inhibitor family that includes tacrolimus, out of cells, resulting in a decreased permeation of drugs into the brain. Thus, PgP is considered part of the blood brain barrier against the transfer of xenobiotics from circulating blood into brain interstitial fluid. Nimodipine has been successfully used to treat central nervous system disorders such as multi-infarct dementia, stroke and subarachnoid haemorrhage. Nimodipine is also a substrate of PgP, which suggested that the transport across the BBB may be modulated by PgP. In a recent study in hypoxia-ischemia-induced brain damage in mice, the PgP inhibitory cyclosporine A markedly enhanced the effect of nimodipine in the central nervous system CSF (Xiao-Dong et al., Acta Pharmacol Sin, 23 (2002), 225-229). In concentrations ranging from 0.1-50 μmol·L⁻¹ cyclosporine, the uptake of nimodipine by primary cultured BCEC cells varied from 2-20-fold greater Zhang et al., Acta Pharmacol Sin, 24 (2003), 903-906). In a follow-on study Zhang et al. demonstrated a dose-depended effect of baicalin and berberine, both of which have beneficial effects on brain ischemic damage and modulation of PgP, increased the transport of nimodipine across the BBB (Zhang et al., Acta Pharmacol Sin, 28 (2007), 573-578), leading to increased CSF concentrations. Overall, it is considered that inhibiting CYP and PgP activity may permit enhanced plasma and CSF drug concentrations as well as perhaps decreasing the intra- and inter-subject variability that is observed in and between patients.

An important aspect of CYP and PgP inhibition is that it may reduce intra- and interindividual pharmacokinetic variability and modulate the effect of additionally co-administered interacting drugs. In the small intestine, drug efflux pumps, most importantly PgP and CYP, form a competitive barrier against the absorption of xenobiotics. The cooperative activities of PgP and CYP were suggested by their shared location in small intestinal enterocytes and the significant overlap in their substrate activities (Wacker et al., Mol Carcinog, 13, (1995), 129-34). Although the mechanisms involved in the functional interaction between CYP and PgP are not fully understood, it is suggested that intestinal efflux pumps, such as PgP, limit and regulate access of drugs to CYP enzymes from being overwhelmed by the high drug concentrations in the intestine (Benet et al., 3 Control Release, 13 (1999), 25-31). Also, suggested is that various active or inactive metabolites may effect efflux (Lampen et al., 3 Pharmacol Exp Ther, 285 (1998), 1104-12). Thus, the release of CYP substrates such as nimodipine or tacrolimus all along the gastrointestinal tract should serve to increase the plasma and CSF bioavailability and reduce the variability in such bioavailability that is observed for both drugs.

Clinically important PgP inhibitors include azole antifungals, cyclosporine, tacrolimus, calcium channel blockers and cancer chemotherapeutics. Tacrolimus is not only a CYP substrate but also a P-glycoprotein (PgP) substrate. While at concentration of up to 1 μM, tacrolimus had little detectable effect on CYP activities, the affinity for PgP is in the 0.1 μM range. In experiments to evaluate the involvement of PgP, Yokogawa et al. compared the pharmacokinetics and tissue distribution of tacrolimus in mdr-1a knockout and wild type mice after oral tacrolimus administration. The blood concentrations were significantly higher with total clearance reduced by 66%. The concentration in brain tissue was 10-fold higher (Yokogawa et al., Pharm Res, 16 (1999), 1213-8). The variability of CYP and PgP activities is not only due to modulation by xenobiotics or endogenous factors, but is also determined by genetic predisposition; with patients have widely different CYP and PgP activities (Lecointre et al., Fundamental and Clinical Pharmacology, 16 (2002), 455-460).

Physical/chemical properties such as low hydrophobicity, poor solubility in gastrointestinal fluids and extensive first-pass effects were generally assumed to reduce the oral bioavailability of drugs. The bioavailability of nimodipine and cyclosporine (marketed formulations—Nimotop®) after their oral administration is poor and variable, ranging from <5% to >35%, averaging approximately 25%. Although intestinal absorption is dependent on intestinal surface area, transit time and, to a lesser extent emulsification, it is now acknowledged that both the active secretion by PgP from enterocytes into the lumen and the intestinal metabolism by CYP play an important role in the bioavailability of many calcium channel blockers, including nimodipine, and calcineurin inhibitors, including tacrolimus and cyclosporine A.

PgP-mediated multidrug resistance (MDR) is proposed to be the mechanism responsible for the failure of chemotherapy in cancer and variability in the bioavailability of several drugs. PgP is a membranous protein and works as energy-dependent effluxes pump that restricts the intracellular accumulation of susceptible drugs. Calcium channel blockers have been shown to sensitize cancer cells to anti-cancer drugs by reversing PgP expression in cell lines. Nimodipine, a lipophilic calcium channel blocker, has been shown to enhance the cytotoxicity of certain anti-cancer drugs (Durmaz et al., Clinical Neurology and Neurosurgery, 101 (1999), 238-244). Therefore, the combination of nimodipine with various anti-cancer drugs that are susceptible to PgP-mediated multidrug resistance may improve the sensitivity of cancer to such drugs.

The following active agents are known to exert an inhibitory effect on the activity of various CYP enzymes or are substrates of CYP and may have the potential to increase the bioavailability of drugs that are substrates of CYP: Cytochrome P450 Substrates: Diltiazem, Nifedipine, Felodipine, Verapamil, Ciclosporin, Tacrolimus, Sirolimus, Cyclophosphamide, Docetaxel, Doxorubicin, Etoposide, Ifosfamide, Paclitaxel, Tamoxifen, Teniposide, Vinblastine, Vindesine, Gefitinib, Benzodiazepines, Flunitrazepam, Midazolam, Alprazolam, Triazolam, Clonazepam, ketoconazole, Itraconazole, Clotrimazole, Amitriptyline, Imipramine, Clomipramine, Erythromycin, Clarithromycin, Citalopram, Fluoxetine, Norfluoxetine, Sertraline, Atorvastatin, Lovastatin, Simvastatin, Sildenafil, Buspirone, Haloperidol, Venlafaxine, Amiodarone, Ethinylestradiol, Kinins, Indinavir, Ritonavir, Saquinavir, Nelfinavir, Mirtazapine, Nefazodone, Pimozide, Reboxetine, Zopiclone, non-nucleoside reverse transcriptase inhibitors, nevirapine, Alfentanil, Budesonide, Donepezil, Esomeprazole, Omeprazole, Finasteride, Glibenclamide, Cisapride, Terfenadine, Toremifene, Phenobarbital, Carbamazepine, Codeine, Dextromethorphan, Digoxin, Ergot alkaloids, Estradiol, Fentanyl, Ivabradine, Levonorgestrel, Lidocaine, Methadone, Mifepristone, Montelukast, Ondansetron, Paracetamol, Quinidine, Quinine, Testosterone, Theophylline, Valproate, Warfarin, Tetrahydrocannabinol, Antipsychotics, Aripiprazole, Risperidone, Ziprasidone, Fluconazole, Ritonavir, Erythromycin, Telithromycin, Clarithromycin, Ketoconazole, Itraconazole, Nefazodone, Bergamottin (constituent of grapefruit juice) and derivatives thereof, Quercetin, Amiodarone, Aprepitant, Cimetidine, Ciprofloxacin, Ciclosporin, Diltiazem, Imatinib, Echinacea, Enoxacin, Ergotamine, Metronidazole, Mifepristone, Efavirenz, Nevirapine, Gestodene, Mibefradil, Saquinavir, Indinavir, Fluoxetine, Star fruit, Piperine, Verapamil and derivatives of the above.

To maintain plasma and cerebrospinal drug concentrations within therapeutic ranges and to avoid side effects associated with high concentrations of drug, a number of drug delivery or controlled release technologies have been developed. The primary technology used in this patent is the use of the Freund Spherex Labo process for producing minispheres and minicapsules as described in U.S. Pat. Nos. 5,882,680 and 6,312,942). Other encapsulation process, such as those developed my Jintan, Inotech or ITAS may be utilized.

### Enhanced Drug Delivery Nimodipine/Tacrolimus Combination

Both Nimodipine and Tacrolimus exhibit a number of drug delivery challenges, both are poorly solubility and demonstrated variable absorption from the intestinal lumen into the bloodstream. Additionally, the bioavailability of both nimodipine and tacrolimus are adversely affected by CYP metabolism and PgP efflux. From a pharmacological perspective, particularly as it applies to neurological diseases, there are potential synergistic benefits to co-administering nimodipine and tacrolimus in a single, controlled release format administration format. From a bioavailability perspective, co-administration has the potential to regulate or inhibit both CYP and PgP resulting in increased plasma concentration of one or both drugs as well as improved BBB passage leading to enhanced CSF concentrations of one or both drugs. Furthermore, when co-administered the regulation of CYP and/or PgP has the potential to reduce the variability observed in plasma and CSF concentration for both nimodipine and tacrolimus. Thus, the present invention describes a mechanism to produce a combination controlled release drug delivery format that addresses solubility and permeability and permits the co-administration of nimodipine and tacrolimus in a more efficient, patient-friendly and pharmacologically synergistic manner.

Nimodipine and tacrolimus are poor water soluble, highly lipophilic drugs. The current administration format for nimodipine requires that it is first made soluble using oils and surfactants and then encapsulated into a large soft gel capsule format. The large soft gel capsule format is not suited to coating with modified release polymers or similar controlled release formulations. Thus, there is a need in the art for a controlled release system for nimodipine that will prevent plasma concentration peaks or troughs and ensure a steady therapeutic plasma concentration is maintained throughout a 24 hour period. The current invention details the development of controlled release minicapsule or minisphere nimodipine formulations that enable sustained release over a 24 hour period to permit once-daily or twice daily administration. Additionally, the current invention permits the development of novel controlled release combination products as potential therapeutics across a range of disease states.

A key innovation enabled by the current invention is the combination of enhanced solubility micronized nimodipine in solid minicapsules or minispheres and solid minicapsules the core of which contains tacrolimus, presolubilised in a lipid-based formulation, both formulations that are further processed to permit concomitant release of both drugs throughout the gastrointestinal tract.

The present invention incorporates a once-daily formulation of tacrolimus that provides a controlled release of a therapeutically effective amount of tacrolimus in combination with a pharmaceutically acceptable carrier(s) or excipient(s). Formulations of Tacrolimus are described in our co-pending PCT/IE/2008/000039, the entire contents of which are herein incorporated by reference. The resulting 24-hour controlled release will enable an improved pharmacokinetic profile leading to a potentially more effective, safer and convenient product. As the invention permits the release of tacrolimus in soluble or readily-soluble form, it thus enables a true once-daily drug formulation, especially for a small molecule drug with poor water-solubility, possibly with limited stability or a short half-life such as tacrolimus, as the drug is absorbed not only in the small intestine but also in the colon. The invention provides an oral drug delivery technology that permits throughout the entire gastrointestinal tract the release of pre- or readily-solubilised drugs in tandem with a controlled release formulation that permits release and absorption in the small intestine, ileum and/or colon of soluble tacrolimus to ensure true once-daily formulations which is a hydrophobic agent that has demonstrated variable bioavailability.

The present invention utilizes micronized nimodipine, encapsulated with solid gelatine based minicapsules or minispheres that are further coated with control release polymers to enable release of nimodipine as the minispheres pass along the gastrointestinal tract.

In addition to nimodipine the following and other calcium channel blockers or derivatives thereof may exert an effect similar to that observed for nimodipine and may therefore be interchangeable: Amlodipine, Benidipine, Felodipine, Nicardipine, Nifedipine, Nilvadipine, Nisoldipine, Nitrendipine, Lacidipine, Lercanidipine. In addition to tacrolimus the calcineurin inhibitors or derivatives thereof may exert an effect similar to that observed for tacrolimus and may therefore be interchangeable: cyclosporine A and sirolimus.

### Nimodipine and Subarachnoid Hemorrhage

Each year, approximately 30,000 Americans and at least as many more worldwide suffer a subarachnoid hemorrhage (SAH) (AHA—American Heart Association). It has been reported that the highest incidence of disability and death occurs within the first 2 weeks following the initial hemorrhage (Arch Neurol. 1987; 44:769-774). The leading cause of morbidity and mortality from subarachnoid hemorrhage is cerebral vasospasm (Stroke. 1984; 15:566-570).

The most commonly prescribed drug post SAH is nimodipine. Nimodipine is a highly lipophilic calcium channel blocker that, once in the bloodstream effectively crosses the blood brain barrier into the brain where it provides damage control. Nimodipine acts to reduce the incidence and severity of neurological deficits resulting from vasospasm in patients who have had a recent SAH and is indicated for the improvement of neurological outcome by reducing the incidence and severity of ischemic deficits in patients with subarachnoid hemorrhage from ruptured intracranial berry aneurysms regardless of their post-ictus neurological condition. Overall, nimodipine improves cognitive function significantly reduces the risk of cerebral infarction and poor outcome in SAH (Br. Med J. 1989; 298:636-642).

As cerebral vasospasm causes increased systolic flow velocities it is recognised as a major complication in aneurismal subarachnoid haemorrhage (SAH) and it may cause delayed ischemic neurological haemorrhage (DIND). Nimodipine improves the clinical outcome following SAH, in particular when administered intravenously. Replacement of oral nimodipine by intravenous nimodipine was associated with a significant reduction of peak systolic flow velocities (PSV) in spastic but not in non-spastic cerebral vessels. Therefore, intravenous but not oral application of nimodipine reduces the severity of cerebral vasospasm following aneurismal SAH. It has been proposed because intravenous administration permits a more constant plasma and cerebrospinal fluid (CSF) concentration of nimodipine that it may prevent induction of spasmodic or reflex cerebral vasospasms (Wessig et al., Society Proceedings/Clinical Neurophysiology 118 (2007) e9-e116. Therefore, a controlled release oral form of nimodipine that permits a more steady-state plasma or CSF concentration, similar to that enabled through intravenous administration, may be therapeutically beneficial.

### Nimodipine and Brain Trauma

In clinical studies, nimodipine has not been shown to be as effective in traumatic brain injury as had been expected. Any beneficial effect of nimodipine in brain trauma might be offset by systemic hypotension and a consequent drop in cerebral perfusion pressure that can occur after its administration, which can have serious adverse effects in traumatic brain injury and in aneurismal subarachnoid haemorrhage (Vergouwen et al., Lancet Neurol, 5 (2006), 993-994). The above mentioned drop in cerebral perfusion pressure is thought to be a consequence of the peak plasma concentrations that are observed following administration of nimodipine using large soft-gel capsules or by administration of a nimodipine solution through a naso-gastric feeding tube. Therefore, an easy to administer, controlled release nimodipine, demonstrating the dual advantage of controlling the plasma and CSF concentration and being easily administered through naso-gastric feeding tubes, may extend the therapeutic utility of nimodipine to traumatic brain injury.

### Nimodipine and Neurodegeneration

In the recent study by the Bonni group, incubation of cerebellar slices with the calcium channel blocker nimodipine increased synaptic dendritic claw formation, which suggests that voltage-gated calcium channel activation inhibits neuronal dendritic claw formation (Shalizi et al., Science, Vol 311, 1012-1017; Flavell et al., Science, Vol 311, 1008-1012). A further endorsement of the role of calcium channel blockers came from a study demonstrating that long-term use of calcium channel blockers was associated with a significantly reduced risk of a Parkinson disease diagnosis (Neurology, April 2008; 70: 1438-1444).

### Role of Calcineurin and Inhibitors in the Brain

Evidence suggests that that calcineurin plays a role in the neuroprotective mechanism of tacrolimus and cyclosporin A, both of which are know to reduce ischemic brain damage. Subchronic pretreatment with equivalent doses of cyclosporine A has been reported to decrease brain edema after middle cerebral artery occlusion (MCAO) (Shiga et al., 1992). The lower potency of cyclosporine A, as compared with tacrolimus, is presumably attributable to low blood-brain barrier permeability (Begley et al., 1990) and its lower affinity for its immunophilin binding site (Liu et al., 1992).

The proposed role of calcineurin inhibitors in the neuroprotective mechanism could involve a number of cellular processes. FKBP12 is an intracellular hydrophobic protein that complexes not only with the ryanodine and IP3 receptor complexes (Timerman et al., 1993; Zhang et al., 1993; Brillantes et al., 1994; Chen et al., 1994; Cameron et al., 1995a) but also interacts with calcineurin (Cameron et al., 1995b). By binding to FKBP12, both tacrolimus and rapamycin disrupt this complex (Cameron et al., 1995b) and interfere with the associated calcineurin (Zhang et al., 1993; Brillantes et al., 1994; Chen et al., 1994; Cameron et al., 1995a,b).

A further possible mode of tacrolimus activity is that it reduces ischemic brain damage by an antiapoptotic mechanism. Activation-induced apoptosis in T and B cell lines is inhibited by tacrolimus (Fruman et al., 1992b; Genestier et al., 1994), and a role for calcineurin in calcium-triggered apoptosis in fibroblasts has been demonstrated (Shibasaki and McKeon, 1995) with evidence that apoptosis plays a key role in brain damage induced by focal cerebral ischemia has been reported (Li et al., 1995a,b; Linnik et al., 1995).

Cyclosporin and tacrolimus, in addition to blocking various enzymes that result in preventing or reducing neuron cells death and thus preventing neurodegeneration also exert an effect through mitochondrial protective mechanisms. The mitochondrial permeability transition (MPT) is considered to represent one of the final events that results in irreversible damage and subsequent cell death (Zoratti and Szabo, Biochem, Biophys. Acta (1995) 1241:139-176). The MPT has been described in a variety of cell systems and occurs as a consequence of oxidative insults and excitotoxicity. It has been reported that the permeability transition also occurs in neurons. This was inferred from experiments using isolated brain mitochondria in the presence of elevated calcium and dissociated neuronal or glial cultures exposed to glutamate or N-methyl-D-aspartate (Schinder et al., (1996) J. Neurosci., 16 (18): 5688-5697). The loss of mitochondrial potential in isolated neurons during hypoxia-hypoglycemia and reperfusion was prevented by cyclosporin A. Additionally, cyclosporine A is a potent inhibitor of the MPT and prevents mitochondrial depolarization induced by N-methyl-D-aspartate, a process that is believed to involve interaction with mitochondrial porin (Bernardi et al., (1994); J. Bioenerg. Biomembr. 16: 509-517). The mitochondrial porin is a demonstrated to have a role in mitochondrial dysfunction and neuronal impairment during ischemia-reperfusion, and indicative that cyclosporine A interaction with porin may be important in neuroprotection attributed to cyclosporine and other calcineurin inhibitors, including tacrolimus.

### Tacrolimus and Cerebral Ischemia

A previous phase II trial to evaluate tacrolimus as a potential neuroprotective agent following stroke proved inconclusive. However, the neurotrophic and neuroregenerative effects of tacrolimus have been established in vivo and in vitro. A Japanese group published data on the evaluation of the neuroprotective effect of tacrolimus in three different animal models of cerebral ischemia (transient and permanent focal ischemia in rats and transient global ischemia in gerbils). In rat models, a significant reduction in ischemic brain damage was observed when tacrolimus (>0.1 mg/kg iv) was administered immediately after the onset of permanent and transient focal ischemia. Similar neuroprotective activity was observed with tacrolimus (1 mg/kg iv) even after delaying administration for up to 2 h after permanent or 1 h after transient focal ischemia. The neuroprotective effect of tacrolimus was still present 2 weeks after transient focal ischemia and 1 week after permanent focal ischemia. After transient global ischemia in gerbils, tacrolimus (1 mg/kg, iv) given immediately after reperfusion also produced long-lasting neuroprotective effects with a protective time-window of 1 to 2 h.

Results presented at the 73rd Annual Meeting of the Japanese Pharmacological Society in March 2000, showed that in three different rodent middle cerebral artery occlusion (MCO) models, 0.32 to 1.0 mg/kg tacrolimus administered iv immediately after occlusion dose-dependently reduced the infarction area. Time studies suggested that the therapeutic time window was between 1 and 2 h. In combination studies with rt-PA in another rat MCO model, tacrolimus (1 mg/kg) and/or the same dose of rt-PA were administered iv 1, 2 or 3 h after occlusion. Both drugs showed neuroprotective effects when administered alone up to 1 h following occlusion, however, a larger efficacy was seen with both drugs in combination, and a significant reduction in brain damage was observed at up to 2 h following occlusion.

Tacrolimus reduced ischemic damage in the rat cortex by up to 70%, and hippocampal damage by more than 80% in mice following a global ischemic insult. In rat studies, tacrolimus increased axonal growth by 20% after peripheral nerve damage. In cell culture, 50 microM tacrolimus completely inhibited increases in APP holoprotein and mRNA caused by PGE2 treatment. This suggests potential benefit neuropathology associated with APP overexpression, such as brain trauma or ischemia. In a monkey model of stroke, a single iv bolus dose of tacrolimus (0.1 mg/kg) administered immediately, or 3 h after middle cerebral artery occlusion, significantly attenuated the reductions of cerebral metabolic rate of oxygen and oxygen extraction function as well as reducing cortical brain damage. In transient focal ischemia models it reduced infarct size by 53% and blocked the increase in peptidyl-prolyl isomerase activity.

Tacrolimus reduces the alterations induced by middle cerebral artery occlusions (including N-terminal phosphorylation of c-Jun, activation of JNK, suppression of ATF-2 and expression of Fas ligands CD95-L and APO-1L), thus reducing infarct size. It appears that the target of tacrolimus's nerve regeneration effect is heat shock protein-56 (HSP-56), which is part of the steroid receptor complex.

Animal studies have shown that tacrolimus may reduce nitric oxide levels in ischemic tissue and may enhance immediate early gene and hsp72 gene expression after ischemia. Studies in the rat presented at the 1998 Society for Neuroscience meeting demonstrated that axonal loss after dorsal root injury was reduced by tacrolimus. Profuse axonal sprouting was also observed, with some axons regenerating 10 mm rostral to the lesion. Other studies presented at the same meeting suggest there is no time limit from the point of nerve injury for the potential clinical use of tacrolimus. Thus, controlled delivered tacrolimus, alone or in combination with nimodipine, has significant potential for the treatment of any of the above conditions.

### Calcineurin Inhibitors and Neurodegeneration

Single neurons form thousands of specialized connections with other neurons called synapses. The number, strength, and specificity of these synaptic connections ultimately determine and regulate brain function. As such, how synaptic connectivity is established during development and how it is modified through life is important in regulating the maintenance of healthy minds or development of neurological or neurodegenerative diseases. Recently, it was demonstrated that calcium influx through N-methyl-D-aspartate (NMDA) receptors as well as through voltage-gated calcium channels activates the phosphatase calcineurin, which dephosphorylates the transcription factor MEF2, permitting it to turn on target genes that mediate synapse disassembly, a potential contributing factor in the development of certain neurodegenerative diseases, including Alzheimer's disease and Parkinson's disease. In this study, incubation of cerebellar slices with cyclosporine A increased synaptic dendritic claw formation, which suggests that calcineurin activation inhibits dendritic claw formation (Shalizi et al., Science, Vol 311, 1012-1017; Flavell et al., Science, Vol 311, 1008-1012) thereby suggesting that calcineurin inhibitors may reverse this negative effect and may promote or maintain dendritic claw formation.

### Combination Calcium Channel Blocker and Calcineurin Inhibitors

It is clear from prior art and clinical observations that calcium channel blockers and calcineurin inhibitors act synergistically in the brain and thus have the potential, either alone or more preferably in combination, to prevent or treat a number of neurological conditions as well as to alleviate damage caused by brain trauma. Additionally, given that both are substrates for CYP and PgP, co-administering calcium channel blockers with calcineurin inhibitors has the potential to enhance the bioavailability of one or both and to reduce inter- and intra-subject bioavailability variability. The current invention enables the co-administration of calcium channel blockers and calcineurin inhibitors such that each drug is released in a sustained manner and in a solubility-enhanced format. In the following section, a number of potential indications for a controlled release, enhanced solubility calcium channel blocker/calcineurin inhibitor combination products is highlighted.

### Combination for Subarachnoid Hemorrhage

Nimodipine, when administered 60 milligrams every 4 hours should be initiated within 96 hours and continued for 21 days, is indicated to reduce the severity of ischemic neurological deficits in patients with subarachnoid hemorrhage including all Hunt and Hess grades [I through V] (Thomson Report, 2005). As tacrolimus is known to prevent or reverse the follow-on effects of ischemic insult, low-dose (1-10 mg/per day) tacrolimus is expected to have a beneficial effect in subarachnoid hemorrhage.

The current invention will permit the development of once-daily or twice daily nimodipine in combination with tacrolimus for the treatment of subarachnoid hemorrhage. In addition to the added convenience of once-daily, the combined minicapsule format will be suited to easy administration through naso-gastric tubing, either with or without need for a funnel-like tube attachment.

### Combination for Stroke/Transient Ischemia

Stroke is the third leading cause of death in the United States and the most common cause of adult disability. An ischemic stroke occurs when a cerebral vessel occludes, obstructing blood flow to a portion of the brain.

The only currently approved medical stroke therapy, tissue plasminogen activator (tPA), is a thrombolytic that targets the thrombus within the blood vessel. Neuroprotective agents, another approach to stroke treatment, have generated as much, interest as thrombolytic therapies.

Ischemia leads to excessive activation of excitatory amino acid receptors, accumulation of intracellular calcium, and release of other toxic products that cause cellular injury. By preventing excitatory neurotransmitter release, neuroprotective agents may reduce deleterious effects of ischemia on cells.

Using various mechanisms, neuroprotective agents attempt to, save ischemic neurons in the brain from irreversible injury. Studies in animals indicate a period of at least 4 hours after onset of complete ischemia in which many potentially viable neurons exist in the ischemic penumbra. In humans, the ischemia may be less complete, and the time window may be longer, but human patients also tend to be older with comorbidities that may limit benefit. As many neuroprotective drugs reduce ischemic damage in animal models of stroke, this line of pharmaceutical research holds great promise.

Known to modulate excessive cellular calcium influx caused by ischemia, calcium channel blockers and calcineurin inhibitor combinations, such as a controlled release nimodipine/tacrolimus combination may prevent neuronal injury.

The current invention will permit the development of once-daily or twice daily nimodipine in combination with tacrolimus for the treatment of stroke, or transient ischemia. In addition to the added convenience of once-daily, the combined minicapsule format will be suited to easy administration through naso-gastric tubing, either with or without need for a funnel-like tube attachment. Additionally, the product may be combined with thrombolytic agents such as tissue plasminogen activator (tPA) or anti-coagulants such as asprin or the like.

### Combination for Brain Trauma

In the pathogenesis of cerebral insufficiency in brain trauma patients the group of most significant factors could be outlined. Usually it involves traumatic injury, hypoxia, ischemia and endotoxemia. Disturbances of calcium metabolism are well documented follow on from trauma. As nimodipine penetrates the BBB, it modulates the permeability of calcium channels and improves cerebral circulation and neuronal activity by binding, the dihydropiridine receptors and has the antioxidant properties. Likewise, tacrolimus passes the BBB and has a positive regulator effect on calcium intracellular levels.

The current invention will permit the development of once-daily or twice daily nimodipine in combination with tacrolimus for the treatment of brain trauma and ischemia. In addition to the added convenience of once-daily, the combined minicapsule format will be suited to easy administration though naso-gastric tubing, either with or without need for a funnel-like tube attachment

### Combination for Neurodegeneration

The above Bonni group study suggested that the beneficial effects of calcium channel blockers and/or calcineurin inhibitors would be of particular benefit to prevent the progression of neurodegenerative diseases such as Parkinson's disease and Alzheimer's disease (Science, February 2006). Co-administration of calcineurin inhibitors and calcium channel blockers such as tacrolimus and nimodipine may exhibit synergistic therapeutic benefits. The additional use of certain essential oils, such as the omega-3 EPA and DHA oils as solubility and permeability enhancers, antioxidants as a preservatives as well as being neuroprotectant may contribute to overall brain health. Thus, such formulations may serve a dual purpose of facilitating the formulation as well as enhancing the therapeutic benefits.

As the current invention will enable the development of safe and convenient once- or twice-daily formats and since both the lipophilic calcium channel blocker nimodipine and the calcineurin inhibitor tacrolimus can be combined into a single hard gelatin pill format, the result will be a dual-action, safe, effective and convenient means to prevent or treat neurodegenerative diseases such as Parkinson's disease and Alzheimer's disease as well as vascular dementia, cognitive impairment and so forth.

### Combination Plus Acetylcholinesterase Inhibitors and Alzheimer's Disease

Co-administration with acetylcholinesterase (AChE) inhibitors or nootropic agents, including tacrolimus or cyclosporine A, should demonstrate a synergistic benefit in Alzheimer's disease treatment. One such AChE inhibitor is Huperzine A, more effective than Tacrine that has been approved in China for the treatment of Alzheimer's disease. It has been suggested that L-calcium channel blockers, through dilating the cerebrovascular vessels, has a positive effect on brain health and function, being particularly beneficial to Alzheimer's disease patients.

As Huperzine A exhibits a similar half-life as nimodipine, it will benefit from being developed as a format enabled by the current invention that will result in a similar controlled release profile being developed. A once-daily dual-action therapeutic, with or without tacrolimus, in a once-daily convenient format has significant potential as a front-line Alzheimer's disease or, indeed, vascular dementia treatment.

Combination with Hydralazine for Ischemic Diseases

Hydralazine is a vasodilator used to treat severe hypertension, congestive heart failure and myocardial infarction. The exact mode of action is unclear but is thought to involve altered calcium ion balance in vascular smooth muscle cells. During treatment of congestive heart failure, concomitant administration of hydralazine with isosorbide dinitrate prevents early development of nitrate tolerance and reduces long-term mortality, suggesting that hydralazine inhibits activation of membrane-associated oxidase which would lead to increased superoxide production. An interesting target of hydralazine is protocollagen prolyl hydroxylase, a downstream target of which is hypoxia-inducible factor-α (HIF-α). Hydralazine has been shown to rapidly and transiently induce HIF-α via inhibition of hydroxylases, to induce VEGF production and stimulate neo-angiogenesis in vivo. Additionally, hydralazine may release nitric oxide indirectly. It is suggested that activation or modulation of HIF-α may be a feasible treatment of ischemic disease (Knowles et al., Circ Res. 2004; 95:162-169). Additionally, NO-donor conjugated hydralazine may be included with or substituted for hydralazine. Combined with nimodipine and/or tacrolimus, hydralazine in a controlled release product, released concurrent or sequentially with nimodipine and/or tacrolimus, has the potential to act as an adjuvant in the prevention or treatment of neural ischemic events or diseases.

Combination with Nitric Oxide Donors

Nitric Oxide (NO) donor, including (Z)-1-[N-(2-aminoethyl)-N-(2-ammonioethyl)amino]diazen-1-ium-1,2-diolate (DETA/NONOate), administration to young adult rats significantly increases cell proliferation and migration in the subventricular zone and the dentate gyrus, neurogenesis in the dentate gyrus as well as increases cell proliferation and migration in the subventricular zone and the dentate gyrus, and these rats exhibit. Significant improvements of neurological outcome during recovery from ischemic stroke. This indicates that nitric oxide is involved in the regulation of progenitor cells and neurogenesis in the adult brain. This suggests that nitric oxide delivered to the brain well after stroke may have therapeutic benefits (Zhang et al., Ann. Neurol. 2001, vol. 50, 5, 602-611). Nitroglycerin has recently been a focus of study as a neuroprotective agent with cerebral vasodilatory, systemic antihypertensive, and neuronal anti-excitotoxic properties. In a phase II trial in 37 acute stroke patients, transdermally administered nitroglycerin lowered blood pressure by 5% to 8% (Bath, Stroke 2002, 33:648-649). Combined with nimodipine and/or tacrolimus, NO-donors in a controlled release product has the potential to act as an adjuvant in the prevention or treatment of neural ischemic events or diseases.

Combination with Statins

Simvastatin, a statin, administration upregulates endothelial nitric oxide synthase (eNOS), resulting in more functional protein, augmentation of cerebral blood flow, and neuroprotection in a murine model of cerebral ischemia. In the present study we used mevastatin to show that the statins as a general class increase eNOS mRNA and protein levels and protect against stroke damage. We also show that mevastatin demonstrates a different potency compared with previously reported statins. By varying the duration of treatment and dosage we were able to establish a drug treatment window, and we determined that tachyphylaxis does not develop after 1 month of daily mevastatin administration. Finally, we determined that enhanced eNOS mRNA and protein expression corresponded to the protective actions of mevastatin in ischemic brain. Mevastatin resulted in an increase in eNOS mRNA and protein levels and augmented absolute CBF. The increased CBF is presumably due to decreased vascular resistance, which may also reflect decreased platelet aggregation and/or leukocyte adhesion by NO-dependent mechanisms (Amin-Hanjani et al., 2001; Stroke; 32:980).

The present invention permits the development of once-daily or twice-daily, sustained release nimodipine and/or tacrolimus alone or in combination with sustained release hydralazine, nitric oxide donors, lipophilic statins, angiotensin II receptor antagonists, anti-coagulants or any combination thereof for the treatment of stroke.

### Neuropathic Pain

Calcium plays an important role in the transmission of pain signals in the central nervous system. At the pre-synaptic nerve terminal, voltage-gated calcium channels open in response to action potential to allow an influx of calcium ions which, in turn, leads to release of various neurotransmitters that diffuse across the synaptic cleft to the post-synaptic membrane to bind to specific receptors. Morphine, is the drug of choice for treatment of chronic pain and bind to opioid receptors on both pre- and post-synaptic membrane receptors which block voltage-gated calcium channels, thereby reducing release of pain producing neurotransmitters such as substance P, thus alleviating pain. It is known that L- and N-type calcium channels are responsible for neurotransmitter release from sensory neurons of the dorsal column of the spinal cord. To capitalize on this, a number of studies have demonstrated an increase in analgesic response of opioids such as morphine, when co-administered with L-type calcium channel blockers (Santillan et al., Pain, 76, 17-26, 1998). Of particular interest is a study investigating co-administration of nimodipine and morphine that indicated an increased analgesic of morphine as well as a longer duration of action at a lower dose (Verma et al., J. Biosci. 30(4), September 2005, 491-497). Other classes of drugs, including compound of the α-aminoamide family with potent Na+ channel blocker properties exhibit long lasting antinociceptive activity and antiallodynic effects in models of chronic pain.

The current invention enables the development of once-daily or twice daily, controlled release nimodipine, with or without co-administered tacrolimus, in combination with a sustained release opiate, including but not limited to morphine, morphine sulphate, tramadol, oxycodone, hydroxycodone, fentanyl, naproxen or NO donor-conjugated naproxen, pregabalin, a sustained release α-aminoamide or any combination thereof for the treatment of neuropathic pain.

### Minicapsule and Minisphere Process

The principle of seamless liquid- or semi-liquid-filled minicapsule or solid minisphere formation is the utilisation of surface tension of one or more different solutions which when ejected through an orifice or nozzle with a certain diameter and subject to specific frequencies and gravitational flow, forms into a spherical form and falls into a cooling air flow or into a cooling or hardening solution and the outer shell solution where it is gelled or solidified. This briefly describes the formation of seamless minispheres.

According to prior art the core solution is mainly a hydrophobic solution or suspension. The outer shell solution can be any gel forming agent but is normally gelatin based but may also include polymers or other materials that enable controlled release. However a hydrophilic solution can also be encapsulated with the existence of an intermediate solution, which can avoid the direct contact of the hydrophilic core solution with the outer shell. With the nozzle having a single orifice, a minicapsule or a bead of shell/core mixed suspension of micronized drug can be processed. With the nozzle having two orifices (centre and outer), a hydrophobic solution can be encapsulated. With the nozzle having one or orifices seamless minicapsules for various applications can be processed. (Ref U.S. Pat. Nos. 5,882,680 and 6,312,942). Additionally, other encapsulation and miniencapsulation processes such as, but not limited to those developed by ITAS (Globex), Innotech, Morishita Jintan, may be utilized.

By using the above described manufacturing processing method as per U.S. Pat. No. 5,882,680 for multiparticulate seamless minicapsules, Nimodipine multiparticulate seamless minicapsules were produced. The completed Nimodipine seamless minicapsules preferably have an average diameter of 1.00-3.00 mm, more especially in the range 1.50-1.80 mm as described in our WO2006/035417A.

The resulting one-, two- or three-layer minicapsules or minispheres may be further processed to be coated with various controlled release polymers which modulates the release of active pharmaceutical actives from the underlying minicapsule or minisphere cores. In accordance with previous inventions the drug loaded minicapsules are coated with the rate-controlling polymers to achieve a target dissolution rate. The drug released from these minicapsules is diffusion controlled as the polymer swells and becomes permeable, it allows for the controlled release in the GIT. In order to achieve a suitable dissolution profile, the following parameters require consideration, efficient process/conditions, drug solubility/particle size, minicapsule surface area, minicapsule diameter and coating polymer suitability.

Additionally, certain semi-solid core formulations may result in controlled release alone or in conjunction with the shell, controlled release shell and/or controlled release shell coating.

As well as minicapsule and minisphere formulated calcium channel blockers and calcineurin inhibitors, other formulation approaches, including, but not limited to drug layering, granulation and melt extrusion may be utilized.

### Controlled Release Polymers—Membrane-Controlled Dosage Forms

The modified-release formulations of the present invention can also be provided as membrane-controlled formulations. Membrane-controlled formulations of the present disclosure can be made by preparing a rapid release core, which can be liquid, semi-solid or solid, encapsulated by a gelatin shell, and coating the shell a functional coating. In the presence or absence of the membrane-controlled coating, the core, whether liquid, semi-solid or solid, can be formulated such that it itself controlled the release rate of the pharmaceutical compound from the minicapsules Details of membrane-controlled dosage forms are provided below.

In certain embodiments of the current invention, the pharmaceutical compound is provided in a multiple minicapsule membrane-controlled formulation. The active pharmaceutical can be formulated as a liquid, semi-solid or solid entity to enhance solubility, permeability or dissolution rate and utilized as the core of a two- or three-layer minicapsule that additionally comprises a shell with or without an additional buffer layer between to separate miscible core and shell constituents. The minicapsule diameter may range from 0.5 to about 5.0 mm. Additional pharmaceutical compound of the same active or one or more other actives can be sprayed from solution or suspension using a fluidized-bed coater or pan coating system.

To control the location of formulation release from the minicapsules, various delayed-release and/or extended-release polymeric materials, applied as a membrane coating to the minicapsules. The polymeric materials include both water-soluble and water-insoluble polymers. Possible water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof. Possible water-insoluble polymers include, but are not limited to, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride), or polyurethane, and/or mixtures thereof.

EUDRAGIT™™ polymers (available from Evonik) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers that are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In various embodiments comprising a membrane-controlled dosage form, the polymeric material comprises methacrylic acid co-polymers, ammonio methacrylate co-polymers, or mixtures thereof. Methacrylic acid co-polymers such as EUDRAGIT™ S and EUDRAGIT™ L (Evonik) are suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material can exhibit solubility at a pH between the pHs at which EUDRAGIT™ L and EUDRAGIT™ S are separately soluble.

The membrane coating can comprise a polymeric material comprising a major proportion (i.e., greater than 50% of the total polymeric content) of at least one pharmaceutically acceptable water-soluble polymers, and optionally a minor proportion (i.e., less than 50% of the total polymeric content) of at least one pharmaceutically acceptable water insoluble polymers. Alternatively, the membrane coating can comprise a polymeric material comprising a major proportion (i.e., greater than 50% of the total polymeric content) of at least one pharmaceutically acceptable water insoluble polymers, and optionally a minor proportion (i.e., less than 50% of the total polymeric content) of at least one pharmaceutically acceptable water-soluble polymer.

The amino methacrylate co-polymers can be combined in any desired ratio, and the ratio can be modified to modify the rate of drug release. For example, a ratio of EUDRAGIT™ RS:EUDRAGIT™ RL of 90:10 can be used. Alternatively, the ratio of EUDRAGIT™ RS:EUDRAGIT™ RL can be about 100:0 to about 80:20, or about 100:0 to about 90:10, or any ratio in between. In such formulations, the less permeable polymer EUDRAGIT™ RS would generally comprise the majority of the polymeric material with the more soluble RL, when it dissolves, permitting creating gaps through which solutes can enter the core and dissolved pharmaceutical actives escape in a controlled manner.

The amino methacrylate co-polymers can be combined with the methacrylic acid co-polymers within the polymeric material in order to achieve the desired delay in the release of the drug. Ratios of ammonio methacrylate co-polymer (e.g., EUDRAGIT™ RS) to methacrylic acid co-polymer in the range of about 99:1 to about 20:80 can be used. The two types of polymers can also be combined into the same polymeric material, or provided as separate coats that are applied to the core.

In addition to the EUDRAGIT™ polymers discussed above, other enteric, or pH-dependent, polymers can be used. Such polymers can include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethylcellulose acetate succinate, starch acetate phthalate, amylose acetate phthalate, polyvinyl acetate phthalate, and polyvinyl butyrate phthalate.

Surelease®, an aqueous ethylcellulose dispersion, is a unique combination of film-forming polymer; plasticizer and stabilizers. Designed for sustained release and taste masking applications, Surelease® is an easy-to-use, totally aqueous coating system using ethylcellulose as the release rate controlling polymer. The dispersion provides the flexibility to adjust drug release rates with reproducible profiles that are relatively insensitive to pH.

The principal means of drug release is by diffusion through the Surelease® dispersion membrane and is directly controlled by film thickness. Increasing or decreasing the quantity of Surelease® applied can easily modify the rate of release.

With Surelease® dispersion, reproducible drug release profiles are consistent right through from development to scale-up and production processes. More information can be found on the Colorcon Inc website at www.Colorcon.com. Additionally, a further range of controlled release polymers may be used.

Additionally, alternative controlled release enabling polymers or other entities may be used alone or in combination with polymers such as those mentioned above, including but not limited to Eudragit™ and Surelease® polymers. Alternatively, any blend of controlled release materials or polymers may be employed.

The coating membrane can further comprise at least one soluble excipient to increase the permeability of the polymeric material. Suitably, the at least one soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose, and sucrose, sugar alcohols such as lactitol, maltitol, mannitol, sorbitol, and xylitol, xanthan gum, dextrins, and maltodextrins. In some embodiments, polyvinyl pyrrolidone, mannitol, and/or polyethylene glycol can be used as soluble excipients. The at least one soluble excipient can be used in an amount ranging from about 1% to about 20% by weight, based on the total dry weight of the polymer. The coating process can be carried out by any suitable means, for example, by using a perforated pan system such as the GLATT, ACCELACOTA, Diosna and/or HICOATER processing equipment.

The modifications in the rates of release, such as to create a delay or extension in release, can be achieved in any number of ways. Mechanisms can be dependent or independent of local pH in the intestine, and can also rely on local enzymatic activity to achieve the desired effect. Examples of modified-release formulations are known in the art and are described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566.

With membrane-modified extended-release dosage forms, a semi-permeable membrane can surround the formulation containing the active substance of interest. Semi-permeable membranes include those that are permeable to a greater or lesser extent to both water and solute. This membrane can include water-insoluble and/or water-soluble polymers, and can exhibit pH-dependent and/or pH-independent solubility characteristics. Polymers of these types are described in detail below. Generally, the characteristics of the polymeric membrane, which may be determined by, e.g., the composition of the membrane, will determine the nature of release from the dosage form.

A number of modified dosage forms suitable for use are described below. A more detailed discussion of such forms can also be found in, for example The Handbook of Pharmaceutical Controlled Release Technology, D. L. Wise (ed.), Marcel Decker, Inc., New York (2000); and also in Treatise on Controlled Drug Delivery: Fundamentals, Optimization, and Applications, A. Kydonieus (ed.), Marcel Decker, Inc., New York, (1992), the relevant contents of each of which are hereby incorporated by reference for this purpose. Examples of modified-release formulations include but are not limited to, membrane-modified, matrix, osmotic, and ion-exchange systems. All of these can be in the form of single-unit or multi-unit dosage forms, as alluded to above.

The pH-dependent systems exploit the generally accepted view that pH of the human GIT increases progressively from the stomach (pH 1-2 which increases to 4 during digestion), small intestine (pH 6-7) at the site of digestion and it increases to 7-8 in the distal ileum. The coating of pH-sensitive polymers to the tablets, capsules or pellets provide delayed release and protect the active drug from gastric fluid. The polymers used for colon targeting, however, should be able to withstand the lower pH values of the stomach and of the proximal part of the small intestine and also be able to disintegrate at the neutral of slightly alkaline pH of the terminal ileum and preferably at the ileocecal junction.

While the minicapsule process above exhibits a number of benefits for a range of active pharmaceutical compounds potential limitations include compatibilities of core formulations with the shell material and/or the buffer layer, where required. Another potential limitation is low active pharmaceutical compound payloads leading to large, patient-unfriendly pill sizes. Still another potential limitation is that controlled release is a function of the shell or shell coating and may thus be limiting. Yet another limitation relates to possible incompatibilities between the shell and the core or the buffer layer which results in incomplete encapsulation or irregular shaped minicapsules. Still another advantage relates to the possibility to develop novel, otherwise incompatible, controlled release combination products for the potential treatment of an array of disease conditions.

### Administration Formats

The multiple minicapsule or minisphere format enables combinations of one active with different controlled release coatings or alternatively different actives with single or multiple controlled release coatings to be filled into hard gelatine capsules of various sizes. The hard gelatine capsule may also contain liquid formulations or powder formulations. Furthermore, the minicapsules or minispheres may be compressed into pellet or pill format comprised or inactive excipients or other active pharmaceutical ingredients.

An advantage of the current minicapsule and minisphere forms is that they are format flexible leading to ease of administration. A common problem in many of the conditions with potential to be treated by nimodipine or combination products containing nimodipine is that patient's experience swallowing difficulties. This may arise due to a patient being incapacitated following a stroke or trauma and fed through a naso-gastric tube or in certain neurodegenerative diseases such as Parkinson's disease where the patient may experience difficulty in swallowing.

In one easy to administer format, the present invention permits that the minicapsules or minispheres may be filled into sachets, the contents of which may be sprinkled onto soft food or, indeed, drinks and administered to patients by spoon feeding, drinking or through a straw. This form of administration is suited to paediatrics or geriatrics that dislike or have difficulty swallowing. Furthermore, the sachet contents may be poured into an attachment to naso-gastric tubing for administration to incapacitated patients. Another format is to pre-fill the contents into a syringe that may be connected to naso-gastric tubing.

Still another administration format is in suppository format that is suited to vaginal or rectal administration. This format has a number of advantages, including administration to patients in acute need for a rapid onset of action and may be incapable of swallowing.

Additionally, the minicapsules or minispheres may be incorporated into a format for buccal or sub-lingual administration. Such formats may include bioadhesive degradable films, including hydrogels or formats that may disintegrate rapidly in the mouth or under the tongue. Again, this format is suited to the need for a quick onset of action or for patients unable to swallow.

### Controlled Release Nimodipine and Tacrolimus Combination

### Uncoated Nimodipine Minicapsules

Appropriate quantities of micronised nimodipine, gelatine and sorbitol are added to water and heated to 80° C., continually stirring until in a homogeneous solution is achieved. The solution is then processed into solid minispheres at an appropriate flow rate and vibrational frequency using the manufacturing processing method described in U.S. Pat. No. 5,882,680 The resulting minispheres are cooled in oil. The cooled minispheres are harvested and centrifuged to remove residual oil and dried overnight in an oven. Nimodipine multiparticulate seamless minicapsules were produced. The completed Nimodipine seamless minicapsules had an average diameter in the range 1.50-1.80 mm

| | |
|---|---|
| Ingredients | % w/w |

Core Composition

| | |
|---|---|
| Nimodipine (Micronised) | 37.5 |
| Gelatin | 56.3 |
| Sorbitol | 6.3 |

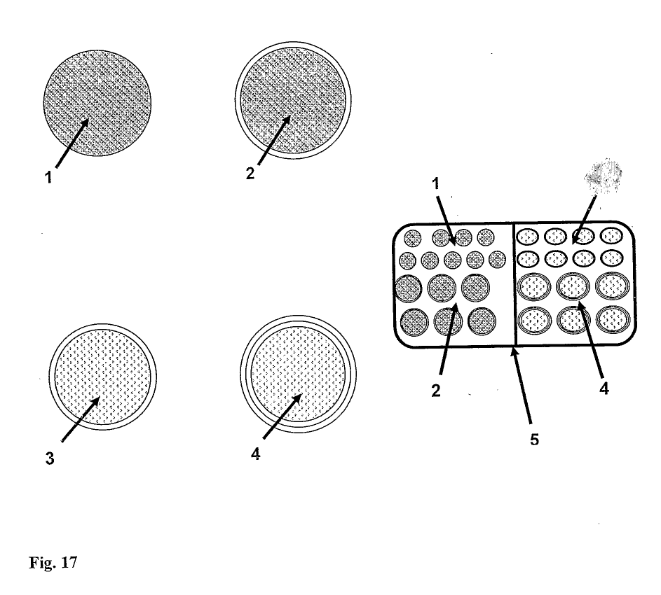

The uncoated nimodipine minicapsules are designated as form 1 in FIG. 17.

### Coated Nimodipine Minicapsules

Some of the uncoated minicapsules are coated with Surelease® using standard bottom spray fluidized bed coating, as enabled using a Diosna Minilab, to provide a 12-hour or a 24-hour release profile.

In one case the coating is a low weight gain Surelease® such as 7.5% wt gain Surelease®, Typically: curing 40° C.×24 hr. The dissolution profile is obtained by placing the resulting minicapsules in 0.3% SDS in Water, 100 rpm, HPLC—over 24 hr.

In another case the coating is a higher weight gain Surelease®, such as 30% wt gain Surelease®, Typically: curing 40° C.×24 hr. The dissolution profile is obtained by placing the resulting minicapsules in 0.3% SDS in Water, 100 rpm, HPLC—over 24 hr.

The uncoated nimodipine minicapsules are designated as form 2 in FIG. 17.

### Uncoated Tacrolimus Minicapsules

The core formulation was prepared as follows. Tacrolimus was dissolved in a suitable volume of ethanol. Once dissolved, the solution was blended with a suitable mix of Labrafil and Olive oil. The shell solution was prepared as follows: Appropriate quantities of gelatin and sorbitol were added to water and heated to 70 degrees C. until in solution. The minicapsules were prepared using a Spherex Labo to produce 2-layer minicapsules, the core of which comprises Tacrolimus in an enhanced solubilised and permeabilised formulation. In addition, the core formulation does enable a degree of sustained release.

| | |
|---|---|
| Ingredients | % w/w |

Core Composition

| | |
|---|---|
| Tacrolimus | 3.25 |
| Labrafil | 36.4 |
| Olive Oil | 47.65 |
| Ethanol | 12.7 |

Shell Composition

| | |
|---|---|
| Gelatin | 90.0 |
| Sorbitol | 10.0 |

The uncoated tacrolimus minicapsules are designated as form 3 in FIG. 17.

### Coated Tacrolimus Minicapsules

Some of the uncoated minicapsules are coated, first with Eudragit™ RS30D (semi-permeable, swellable polymer coating) followed by Eudragit™ FS30D (enteric, pH sensitive coating) using standard bottom spray fluidized bed coating, as enabled using a Diosna Minilab, to provide up to a 24-hour release profile.

The first coating is a 12.5% weight gain Eudragit™ RS30D followed by curing at 40° C. for 24 hr. Once dried, the second coating is a 25% weight gain Eudragit™ FS30D followed by curing at 40° C. for 24 hr. The dissolution profile is obtained by placing the resulting minicapsules in 0.3% SDS in Water, 100 rpm, HPLC—over 24 hr.

The coated tacrolimus minicapsules are designated as form 4 in FIG. 17.

### Final Nimodipine and Tacrolimus Dosage Form

The uncoated nimodipine minispheres and one or more populations of coated nimodipine minispheres are blended and filled into the final dosage form. The uncoated tacrolimus minicapsules and one or more populations of coated tacrolimus minicapsules are blended and filled into the final dosage form. Alternatively, the uncoated nimodipine minispheres and one or more populations of coated nimodipine minispheres are mixed with uncoated tacrolimus minicapsules and one or more populations of coated tacrolimus minicapsules, blended and filled into the final dosage form.

In more detail, FIG. 17 illustrates schematically a population of individual solid, gelatine-based uncoated minispheres 1 encapsulating the micronized nimodipine. In the case illustrated there is a blend of two populations of variably weight-gain Surelease® polymer coated minispheres, 7.5% weight gain and 30% weight gain represented by 2. Also represented is the tacrolimus minicapsules, the uncoated minicapsules 3 encapsulate solubilised tacrolimus in a liquid lipid formulation, the coated minicapsules 4 are gelatine encapsulated solubilised tacrolimus in a liquid lipid formulation that have been coated first with 12.% weight gain Eudragit™ RS30D followed by a 25% weight gain Eudragit™ FS30D coating. The individual uncoated nimodipine minispheres 1, Surelease® coated nimodipine minispheres 2, uncoated tacrolimus minicapsules 3 and Eudragit™ RS30D/Eudragit™ FS30D coated tacrolimus minicapsules 4, are blended and filled into the final dosage form, in this instance, a two-cap, hard gelatine capsule 5.

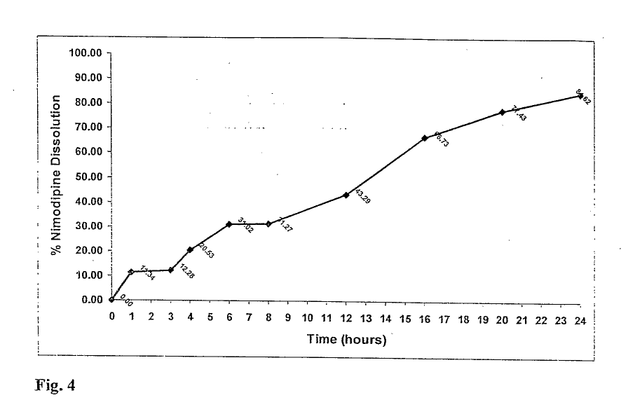

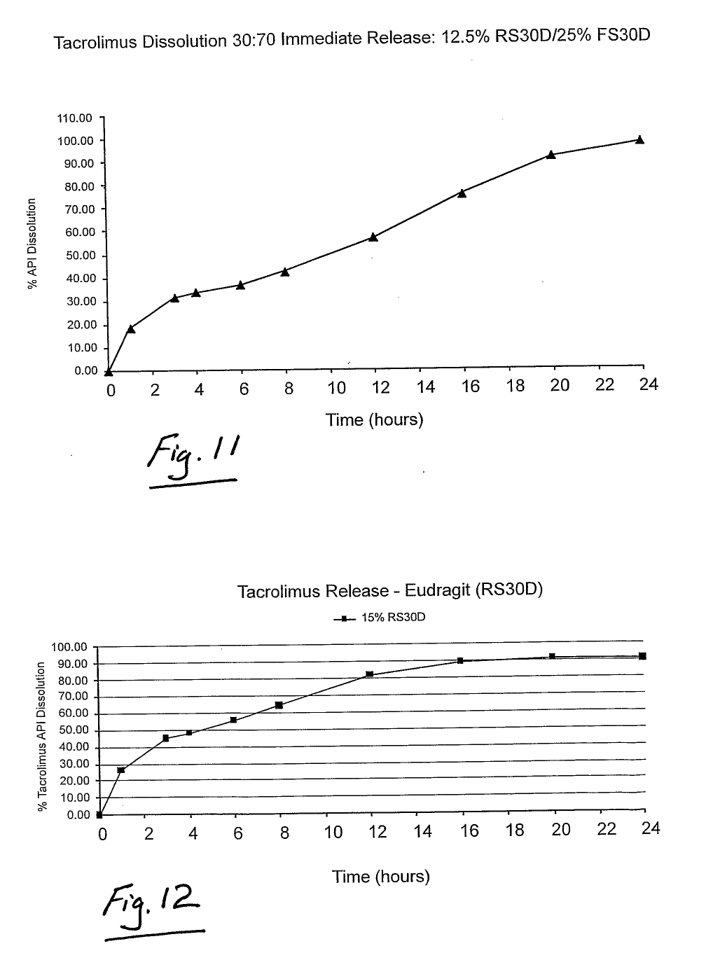

24-hour nimodipine dissolution data is presented in Table 4 and the dissolution profile is graphically illustrated in FIG. 4 with the 24-hour tacrolimus dissolution data presented in Table 9 and graphically illustrated in FIG. 11.

### Example 1

### Nimodipine QD1 Formulation

Using the manufacturing process described above a nimodipine QD1 formulation (30 mg) was prepared from a blend of 5 mg Uncoated, 6 mg 15% wt gain, 19 mg 30% wt gain Surelease, Curing 40° C.×24 hr. The dissolution profile is obtained by placing the resulting minicapsules in 0.3% SDS in Water, 100 rpm, HPLC—over 24 hr.

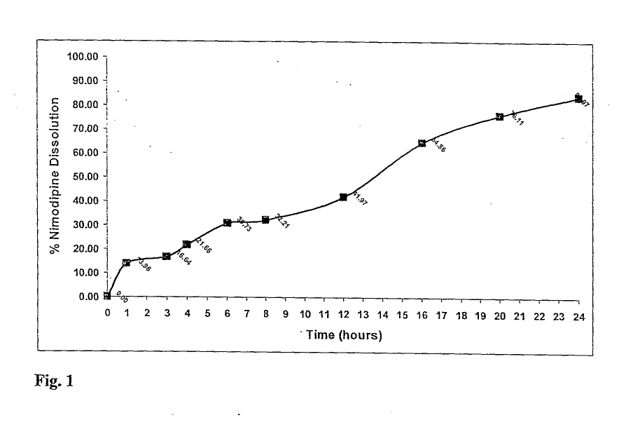

TABLE 1
Release of Nimodipine QD1 Formulation (30 mg) - Blend of
5 mg Uncoated, 6 mg 15% wt gain, 19 mg 30% wt gain
Surelease, Curing 40° C. × 24 hr. The dissolution
profile is obtained by placing the resulting minicapsules in
0.3% SDS in Water, 100 rpm, HPLC - over 24 hr.
The release profile is illustrated in FIG. 1.

| | | |
|---|---|---|
| | Dissolution: | |
| Time | % Release | Average |

| | | | |
|---|---|---|---|
| 0 | 0.00 | 0.00 | 0.00 |
| 1 | 14.88 | 13.04 | 13.96 |
| 3 | 15.80 | 17.47 | 16.64 |
| 4 | 20.52 | 22.79 | 21.66 |
| 6 | 29.75 | 31.70 | 30.73 |
| 8 | 32.44 | 31.97 | 32.21 |
| 12 | 43.34 | 40.60 | 41.97 |
| 16 | 64.13 | 65.58 | 64.86 |
| 20 | 73.86 | 78.36 | 76.11 |
| 24 | 80.56 | 87.37 | 83.97 |

### Example 2

### Nimodipine BID 1 Formulation

Using the manufacturing process described above a nimodipine BID 1 formulation (30 mg) was prepared from a blend of 9 mg uncoated, 21 mg 15% wt gain Surelease, Curing 40° C.×24 hr. The dissolution profile is obtained by placing the resulting minicapsules in 0.3% SDS in Water, 100 rpm, HPLC—over 24 hr.

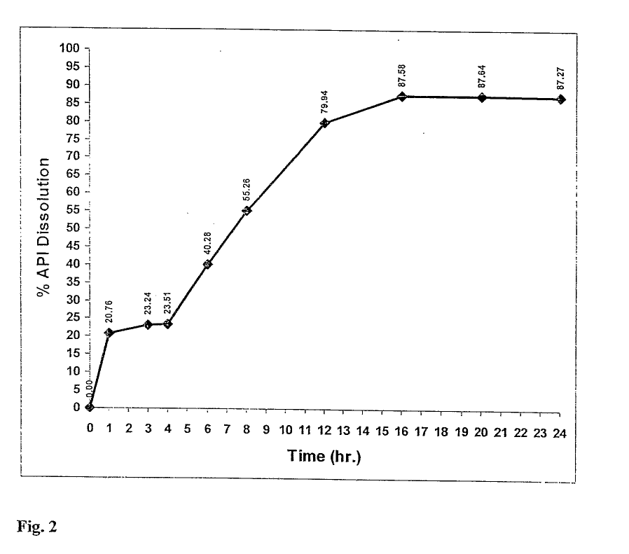

TABLE 2
Release of Nimodipine BID 1 Formulation
(30 mg) - Blend of 9 mg Uncoated, 21 mg
15% wt gain Surelease, Curing 40° C. × 24 hr.
The dissolution profile is obtained by placing the resulting
minicapsules in 0.3% SDS in Water, 100 rpm, HPLC - over 24 hr.
The release profile is illustrated in FIG. 2.

| | | |
|---|---|---|
| | Dissolution | |
| Time | % Release | Average |

| | | | |
|---|---|---|---|
| 0 | 0.00 | 0.00 | 0.00 |
| 1 | 21.76 | 19.75 | 20.76 |
| 3 | 22.64 | 23.84 | 23.24 |
| 4 | 23.11 | 23.90 | 23.51 |
| 6 | 42.63 | 37.93 | 40.28 |
| 8 | 55.93 | 54.58 | 55.26 |
| 12 | 80.17 | 79.71 | 79.94 |
| 16 | 85.69 | 89.47 | 87.58 |
| 20 | 86.16 | 89.12 | 87.64 |
| 24 | 85.24 | 89.30 | 87.27 |

### Example 3

### Nimodipine BID 1 Formulation

Using the manufacturing process described above a nimodipine BID 1 formulation (30 mg) was prepared from a blend of 9 mg Uncoated, 21 mg 20% wt gain Surelease, Curing 40° C.×24 hr. The dissolution profile is obtained by placing the resulting minicapsules in 0.3% SDS in Water, 100 rpm, HPLC—over 24 hr.

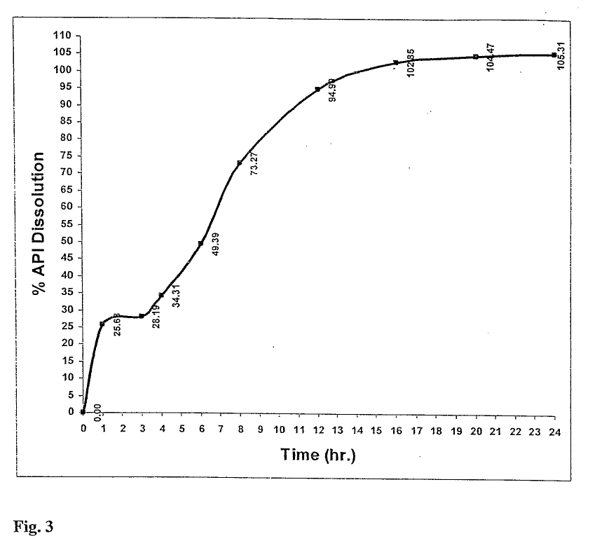

TABLE 3
Release of Nimodipine BID 1 Formulation (30 mg) - Blend
of 9 mg Uncoated, 21 mg 20% wt gain Surelease,
Curing 40° C. × 24 hr. The dissolution profile is
obtained by placing the resulting minicapsules in 0.3% SDS in
Water, 100 rpm, HPLC - over 24 hr. The release profile is
illustrated in FIG. 3.

| | | |
|---|---|---|
| | Dissolution: | |
| Time | % Release | Average |

| | | | |
|---|---|---|---|
| 0 | 0.00 | 0.00 | 0.00 |
| 1 | 27.28 | 27.08 | 22.67 |
| 3 | 27.86 | 28.78 | 27.94 |
| 4 | 33.95 | 36.65 | 32.33 |
| 6 | 49.62 | 55.94 | 42.60 |
| 8 | 72.78 | 80.74 | 66.29 |
| 12 | 96.66 | 96.66 | 91.39 |
| 16 | 101.87 | 104.32 | 102.37 |
| 20 | 104.38 | 104.38 | 104.65 |
| 24 | 106.13 | 105.36 | 104.44 |

### Example 4

### Nimodipine QD1 Formulation

Using the manufacturing process described above a nimodipine QD1 formulation (30 mg) was prepared from a blend of 14.9 mg uncoated, 35.6 mg 7.5% wt gain Surelease®, 130.5 mg 30% wt gain Surelease®, Curing 40° C.×24 hr. The dissolution profile is obtained by placing the resulting minicapsules in 0.3% SDS in Water, 100 rpm, HPLC—over 24 hr. The individual uncoated minispheres 1, lower weight gain Surelease® coated minispheres 2 and higher weight gain Surelease™ coated minispheres 3, are blended and filled into the final dosage form, in this instance, a two-cap, hard gelatine capsule 4, as illustrated in FIG. 9.

TABLE 4
Release of Nimodipine QD Formulation (180 mg) - Blend
of 14.9 mg Uncoated, 35.6 mg 7.5% wt gain
Surelease ®, 130.5 mg 30% wt gain Surelease ®,
Curing 40° C. × 24 hr. The dissolution profile is
obtained by placing the resulting minicapsules in 0.3% SDS in
Water, 100 rpm, HPLC - over 24 hr. The release profile
is illustrated in FIG. 4.

| | Dissolution: | | | |
|---|---|---|---|---|
| Time | % Release | | | Average |
| 0 | 0.00 | 0.00 | 0.00 | 0.00 |
| 1 | 12.03 | 11.03 | 10.96 | 11.34 |
| 3 | 12.04 | 11.96 | 12.85 | 12.28 |
| 4 | 19.46 | 20.24 | 21.90 | 20.53 |
| 6 | 30.67 | 30.43 | 31.95 | 31.02 |
| 8 | 30.81 | 30.79 | 32.21 | 31.27 |
| 12 | 41.21 | 42.02 | 46.63 | 43.29 |
| 16 | 65.53 | 65.81 | 68.86 | 66.73 |
| 20 | 76.04 | 78.34 | 77.91 | 77.43 |
| 24 | 85.10 | 84.03 | 84.72 | 84.62 |

### Example 5

### Controlled Release Nimodipine Human Study

A test product—a single capsule containing 180 mg Nimodipine as per example 4 was administered to healthy male volunteers. The results were compared against administration of 6×30 mg known formulations of nimodipine—Nimotop™ over a 24 hour period. The pharmacokinetic study represented the average of 20 healthy male volunteers and the plasma concentration was measured in ng/ml.

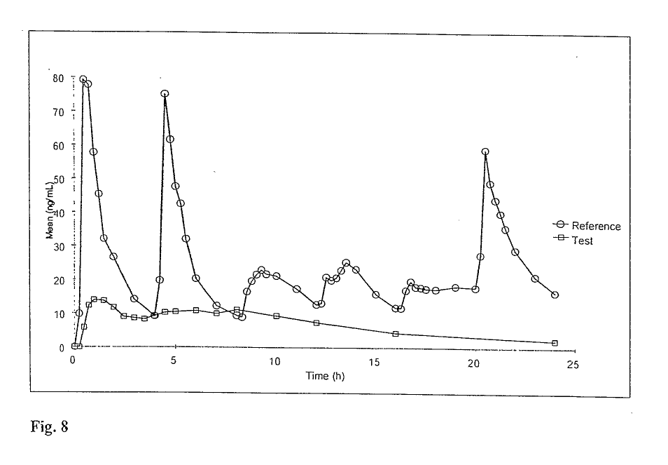

FIG. 8 illustrates the pharmacokinetic plasma profile for the test product (180 mg Nimodipine as per example 4 versus 6×30 mg Nimotop™ over a 24 hour period. The pharmacokinetic study represents the average of 20 healthy male volunteers and the plasma concentration is measured in ng/ml.

This product profile is suited to once- or twice-daily administration.

### Example 6

### Controlled Release Three-Layer Nimodipine Minicapsules

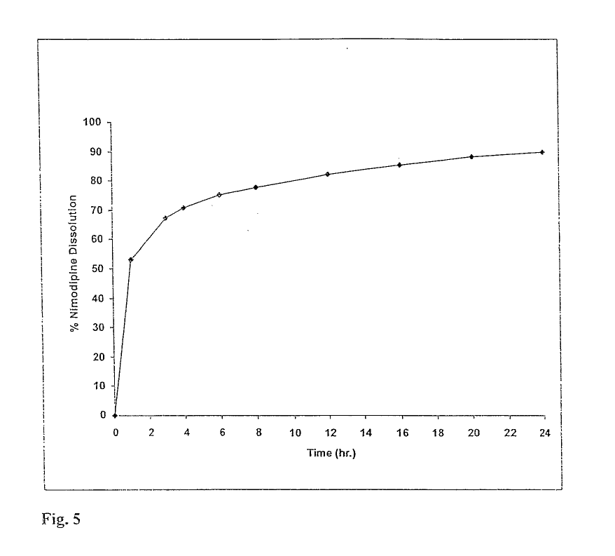

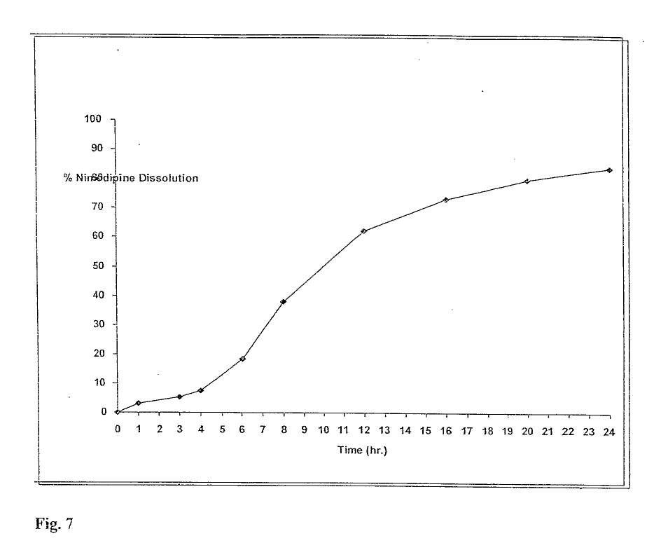

An appropriate quantity of nimodipine is added to PEG 400, heated and stirred until the nimodipine is fully dissolved. The solution is then processed to flow through the central nozzle of a tri-centric nozzle with heated gelucire passing through the middle nozzle and a molten gelatine/sorbitol solution passed through the outer nozzle. The three solutions are passed through the tri-centric nozzle with each flowing at appropriate flow rates and vibrational frequency. The resulting three-layer minicapsules are cooled in oil. The cooled minispheres are harvested and centrifuged to remove residual oil and dried overnight in an oven. The resulting minicapsules are further coating with either a 6.5% or 13.5% weight gain 50:50 Eudragit® RS/Eudragit® RL to provide a 24-hour release profile. The uncoated 3-layer nimodipine 24 hour dissolution data is presented in Table 5 and the related dissolution profile is graphically illustrated in FIG. 5. The Nimodipine 3 Layer Formulation 6.5% weight gain 50:50 Eudragit RS/RL 24 hour dissolution data is presented in Table 6 and the related dissolution profile is graphically illustrated in FIG. 7. The Nimodipine 3 Layer Formulation 13.5% weight gain 50:50 Eudragit RS/RL 24 hour dissolution data is presented in Table 7 and the related dissolution profile is graphically illustrated in FIG. 7.

| | |
|---|---|
| Ingredients | % w/w |

Core Composition

| | |
|---|---|
| Nimodipine | 8.5 |
| PEG 400 | 91.5 |

Mid-Layer Composition

| | |
|---|---|
| Gelucire 33/1 | 100 |

Shell Composition

| | |
|---|---|
| Sorbitol | 10 |
| Gelatin | 90 |

TABLE 5
Nimodipine 3 Layer Formulation Uncoated
(30 mg) - 0.3% SDS in Water, 100 rpm,
HPLC - over 24 hr. The release profile is illustrated in FIG. 5.

| Dissolution (%) | | | |
|---|---|---|---|
| Time (hours) | N = 1 | N = 2 | Average |
| 0 | 0.00 | 0.00 | 0.00 |
| 1 | 53.29 | 52.88 | 53.09 |
| 3 | 67.96 | 66.94 | 67.45 |
| 4 | 71.09 | 70.42 | 70.76 |
| 6 | 75.56 | 74.78 | 75.17 |
| 8 | 78.13 | 77.84 | 77.99 |
| 12 | 82.54 | 82.18 | 82.36 |
| 16 | 85.32 | 85.39 | 85.36 |
| 20 | 87.96 | 88.35 | 88.16 |
| 24 | 89.59 | 90.43 | 90.01 |

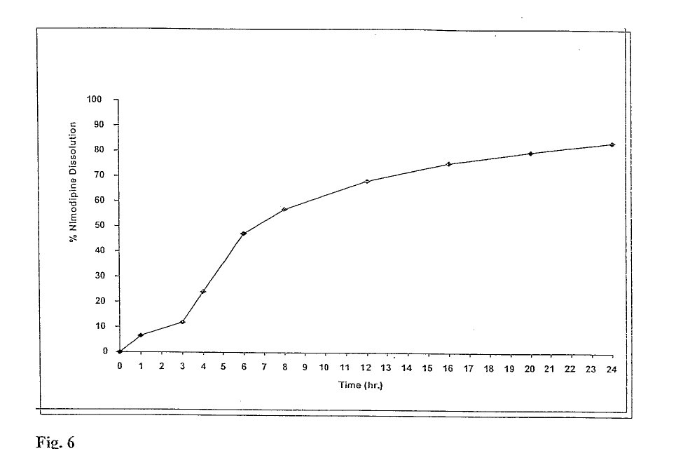

TABLE 6
Nimodipine 3 Layer Formulation 6.5% wt gain 50:50
Eudragit RS/RL (30 mg) - Curing 40° C. × 24 hr,
0.3% SDS in Water, 100 rpm, HPLC - over 24 hr. The
release profile is illustrated in FIG. 6.

| Dissolution (%) | | | |
|---|---|---|---|
| Time (hours) | N = 1 | N = 2 | Average |
| 0 | 0.00 | 0.00 | 0.00 |
| 1 | 6.84 | 6.04 | 6.44 |
| 3 | 12.71 | 10.85 | 11.78 |
| 4 | 24.98 | 23.04 | 24.01 |
| 6 | 47.73 | 46.89 | 47.31 |
| 8 | 55.68 | 58.12 | 56.90 |
| 12 | 66.48 | 69.60 | 68.04 |
| 16 | 73.04 | 77.28 | 75.16 |
| 20 | 77.92 | 81.75 | 79.84 |
| 24 | 81.06 | 85.65 | 83.36 |

TABLE 7
Nimodipine 3 Layer Formulation 13.5% wt gain 50:50 Eudragit
RS/RL (30 mg) - Curing 40° C. × 24 hr,
0.3% SDS in Water, 100 rpm, HPLC - over 24 hr. The release
profile is illustrated in FIG. 7.

| Dissolution (%) | | | |
|---|---|---|---|
| Time (hours) | N = 1 | N = 2 | Average |
| 0 | 0.00 | 0.00 | 0.00 |
| 1 | 3.07 | 3.41 | 3.24 |
| 3 | 5.06 | 5.74 | 5.40 |
| 4 | 6.61 | 8.60 | 7.61 |
| 6 | 14.75 | 22.26 | 18.51 |
| 8 | 35.72 | 40.37 | 38.05 |
| 12 | 61.11 | 63.28 | 62.20 |
| 16 | 72.63 | 73.70 | 73.17 |
| 20 | 78.72 | 80.65 | 79.69 |
| 24 | 82.93 | 84.46 | 83.70 |

### Example 6

### Controlled Release Nimodipine

### With Vitamin E for Enhanced Bioavailability

Appropriate quantities of micronised nimodipine, gelatine, sorbitol and vitamin E are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using Surelease® to provide a 12-hour or a 24-hour release profile.

| | |
|---|---|
| Ingredients | % w/w |

Core Composition

| | |
|---|---|
| Nimodipine | 37.5 |
| Vitamin E | 4.7 |
| Gelatin | 51.6 |
| Sorbitol | 6.3 |

### Example 7

### Once-Daily Tacrolimus

The core formulation was prepared as follows. Tacrolimus was dissolved in a suitable volume of ethanol. Once dissolved, the solution was blended with a suitable mix of Labrafil and Olive oil. The shell solution was prepared as follows: Appropriate quantities of gelatin and sorbitol were added to water and heated to 70 degrees C. until in solution. The minicapsules were prepared using a Spherex Labo to produce 2-layer minicapsules, the core of which comprises Tacrolimus in an enhanced solubilised and permeabilised formulation. In addition, the core formulation does enable a degree of sustained release.

TABLE 8
Once-daily Tacrolimus

| | |
|---|---|
| Ingredients | % w/w |

Core Composition

| | |
|---|---|
| Tacrolimus | 3.25 |
| Labrafil | 36.4 |
| Olive Oil | 47.65 |
| Ethanol | 12.7 |

Shell Composition

| | |
|---|---|
| Gelatin | 90.0 |
| Sorbitol | 10.0 |

### Example 8

Tacrolimus release from uncoated minicapsules of Example 7: Dissolution profiles in FIG. 9 demonstrate the following release of tacrolimus from minicapsules expressed as a percentage of the total minicapsule content: less than 55% within 1 hr; less than 80% within 4 hrs; less than 90% within 12 hrs and less than or equal to 100% at 24 hr.

### Example 9

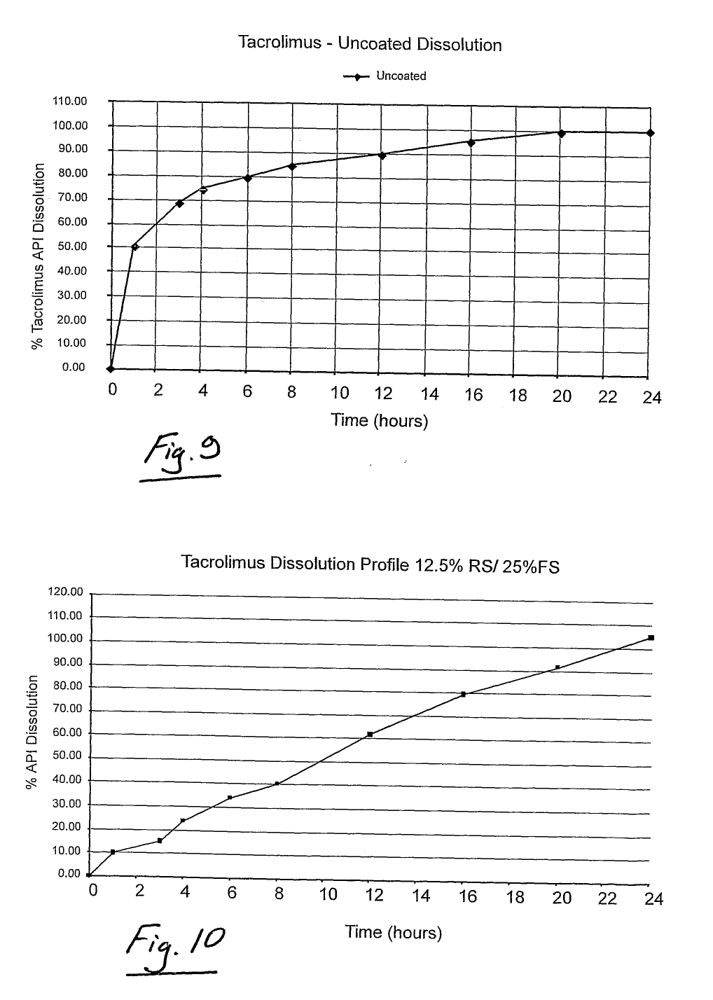

Tacrolimus release from minicapsules of Example 7 coated with 12.5% weight gain Eudragit™ RS30D followed by 25% weight gain Eudragit™ FS30D: Dissolution profiles in FIG. 10 demonstrate the following release of tacrolimus from minicapsules expressed as a percentage of the total minicapsule content: less than 10% within 1 hr; less than 30% within 4 hrs; less than 75% within 12 hrs and less than or equal to 100% at 24 hr. This is suited either to a once-daily systemic absorption product or an ileum/colon-specific product.

### Example 10

Tacrolimus release from a composite of minicapsules of Example 7 comprising 30% (by potency) uncoated and 70% (by potency) coated with 12.5% weight gain Eudragit™ RS30D followed by 25% weight gain Eudragit™ FS30D: Dissolution profiles in FIG. 11 demonstrate the following release of tacrolimus from minicapsules expressed as a percentage of the total minicapsule content: less than 20% within 1 hr; less than 35% within 4 hrs; less than 65% within 12 hrs and less than or equal to 100% at 24 hr.

### Example 11

Tacrolimus release from minicapsules of Example 7 coated with 15% weight gain Eudragit™ RS30D: Dissolution profiles in FIG. 12 demonstrate the following release of tacrolimus from minicapsules expressed as a percentage of the total minicapsule content: less than 30% within 1 hr; less than 50% within 4 hrs; less than 85% within 12 hrs and less than or equal to 100% at 24 hr.

### Example 12

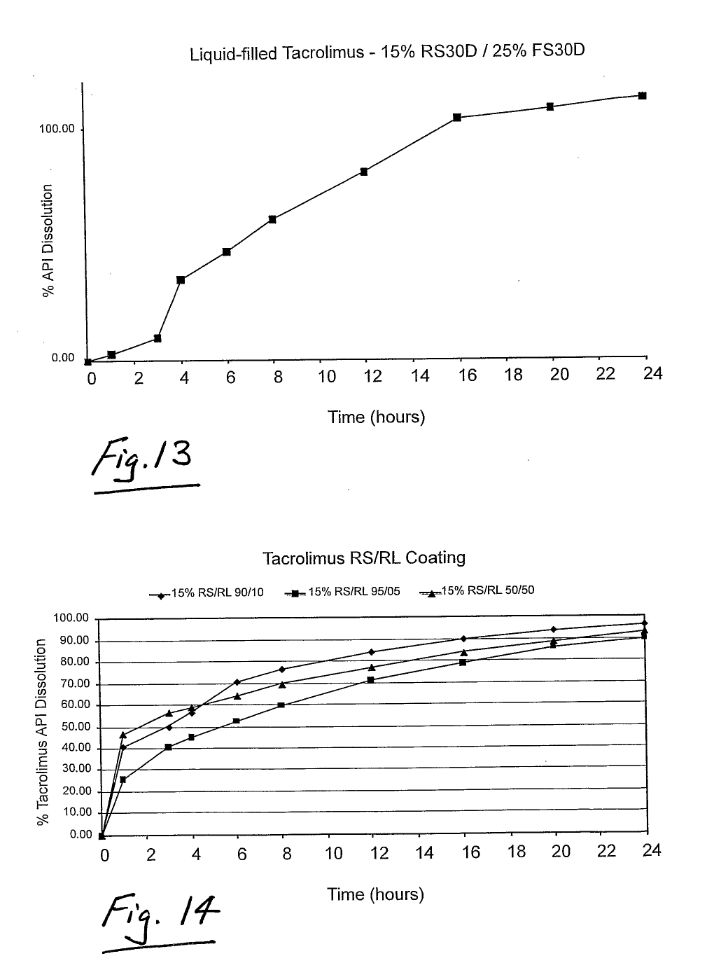

Tacrolimus release from a minicapsules of Example 7 coated with 15% weight gain Eudragit™ RS30D followed by 25% weight gain Eudragit™ FS30D: Dissolution profiles in FIG. 13 demonstrate the following release of tacrolimus from minicapsules expressed as a percentage of the total minicapsule content: less than 10% within 1 hr; less than 30% within 4 hrs; less than 75% within 12 hrs and less than or equal to 100% at 24 hr. This is suited either to a once-daily systemic absorption product or, more particularly, an ileum/colon-specific product.

TABLE 9
Table 9 Tacrolimus 2 Layer Formulation (5 mg Tacrolimus):
The Uncoated minicapsule: Coated Minicapsule API
ratio is 3:7. Coating minicapsules comprise undercoat of
12.5% weight gain Eudragit ™ RS30D and outer
coat of 25% weight gain Eudradgit ™ FS30D,
cured at 40° C. for 24 hr. The formulation dissolution
was measured in 0.3% SDS in Water solution, 100 rpm,
HPLC - over 24 hr. The release profile is illustrated in FIG. 11.

| | Average |
|---|---|
| | Dissolution; |
| | % API |
| | Released |
| Time (Hr.) | (n = 6) |
| 0 | 0 |
| 1 | 18 |
| 3 | 31 |
| 4 | 34 |
| 6 | 37 |
| 8 | 43 |
| 12 | 57 |
| 16 | 76 |
| 20 | 91 |
| 24 | 98 |

### Example 13

Tacrolimus release from minicapsules of Example 7 coated with a combination of Eudragit™ RS and Eudragit™ RL in the following ratios—90:10, 95:05 and 50:50: Dissolution profiles in FIG. 14 demonstrate the following release of tacrolimus from minicapsules expressed as a percentage of the total minicapsule content: greater than 20% and less than 50% within 1 hr; greater than 35% and less than 60% within 4 hrs; greater than 65% and less than 90% within 12 hrs and greater than 90% at 24 hr.

### Example 14

### Once-Daily Tacrolimus

The core formulation was prepared as follows: Tacrolimus was added to a suitable volume Gelcuire 33/01 heated and stirred until dissolved. Once dissolved, the solution was blended with a suitable volume of Olive oil.

The shell solution was prepared as follows: Appropriate quantities of gelatin and sorbitol were added to water and heated to 70 degrees C. until in solution.

The minicapsules were prepared using a Spherex Labo to produce 2-layer minicapsules, the core of which comprises Tacrolimus in an enhanced solubilised and permeable formulation. In addition, the core formulation is inherently sustained release.

TABLE 10
Once-daily Tacrolimus

| | |
|---|---|
| Ingredients | % w/w |

Core Composition

| | |
|---|---|
| Tacrolimus | 0-25 |
| Gelucire 33/01 | 0-75 |
| Olive Oil | 0-75 |
| Ethanol | 0-20 |

Shell Composition

| | |
|---|---|
| Gelatin | 90.0 |
| Sorbitol | 10.0 |

The sustained release coating comprises a 95:5 ratio of Eudragit™ RS:Eudragit™ RL. The combination comprises 95:5 Eudragit™ RS:RL, further coated with Eudragit FS30D.

### Example 15

### Combination Controlled Release Hydralazine

Hydralazine was added to a suitable solution of olive oil, Gelucire 44/01 and Labrafil 1944 heated and continually stirred until in solution. An appropriate amount of gelatine was heated and when in solution was homogenised with the hydralazine solution. The combined mix was passed through a vibrating nozzle to produce 1-layer minicapsules, the core of which comprises Hydralazine in an enhanced solubilised and permeabilised formulation. The resulting minicapsules may be further coated with a 23% weight gain of Eudragit RS30D to enable appropriate controlled release profile to maximise therapeutic benefits.

TABLE 11
Hydralazine Minispheres

| | |
|---|---|
| Ingredients | % w/w |

Core Composition

| | |
|---|---|
| Hydralazine | 4.25 |
| Gelatin | 79.8 |
| Gelucire | 5.32 |
| Olive Oil | 6.11 |
| Labrafil MS 1944 | 4.52 |

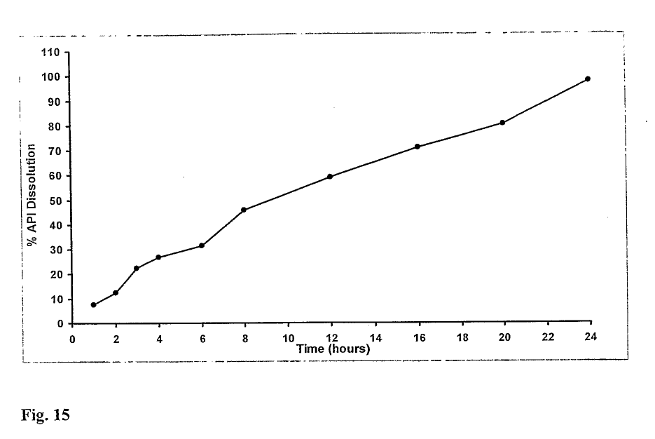

TABLE 12
Hydralazine Minisphere Formulation (As per Example 15) coated
with 23% weight gain Eudragit ™ RS30D - 0.3% SDS in
Water, 100 rpm, HPLC - over 24 hr. The release profile is
illustrated in FIG. 15.

| | Dissolution: % |
|---|---|
| Time | Hydralazine |
| (Hours) | Released |
| 0 | 0 |
| 1 | 7.59 |
| 2 | 12.45 |
| 3 | 22.45 |
| 4 | 26.87 |
| 6 | 31.45 |
| 8 | 45.67 |
| 12 | 58.65 |
| 16 | 70.63 |
| 20 | 80.09 |
| 24 | 97.33 |

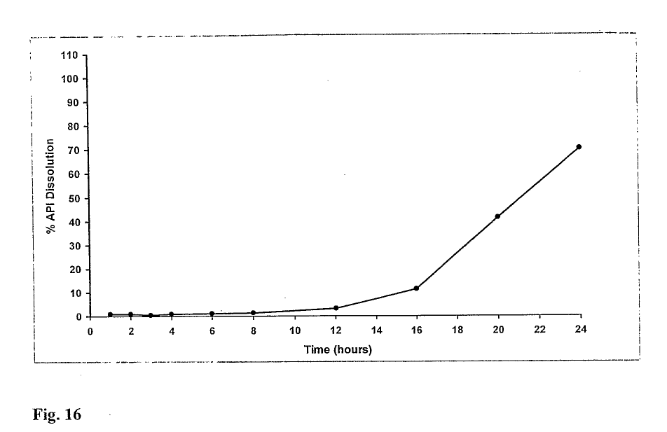

TABLE 13
Hydralazine Minisphere Formulation (As per Example 15) coated
with 30% weight gain Eudragit ™ RS30D - 0.3% SDS in Water,
100 rpm, HPLC - over 24 hr. The release profile is illustrated in FIG. 16.

| | Dissolution: % |
|---|---|
| Time | Hydralazine |
| (Hours) | Released |
| 0 | 0 |
| 1 | 0.87 |
| 2 | 0.93 |
| 3 | 0.50 |
| 4 | 0.96 |
| 6 | 1.07 |
| 8 | 1.36 |
| 12 | 3.00 |
| 16 | 11.42 |
| 20 | 41.19 |
| 24 | 69.92 |

### Example 16

### Combination Controlled Release Nimodipine/Hydralazine

The core formulation was prepared as follows. Hydralazine was dissolved in a suitable volume of ethanol. Once dissolved, the solution was blended with a suitable mix of gelucire. The shell solution was prepared as follows: Appropriate quantities of Eudragit RS, Eudragit RL, micronised nimodipine and sorbitol were mixed and heated to 120 degrees C. until in solution. The minicapsules were prepared using di-centric nozzles to produce 2-layer minicapsules, the core of which comprises Hydralazine in an enhanced solubilised and permeabilised formulation while the shell contains micronised nimodipine. The resulting minicapsules may be further coated to enable appropriate controlled release profile, either concomitant or sequential release, to maximise therapeutic benefits.

| | |
|---|---|
| Ingredients | % w/w |

Core Composition

| | |
|---|---|
| Hydralazine | 2.5-50 |
| Gelucire | 30-50 |
| Olive Oil | 30-50 |
| Ethanol | 0-12.5 |

Shell Composition

| | |
|---|---|
| Eudragit RS | 20-40 |
| Nimodipine (micronised) | 30-40 |
| Eudragit RL | 20-40 |

### Example 17

### Controlled Release Combination Nimodipine and Morphine Sulphate

Appropriate quantities of micronised nimodipine, morphine sulphate, fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| Morphine Sulphate | 20-40 |
| Fumaric Acid | 0-5 |
| Gelatine | 20-50 |
| Sorbitol | 0-10 |

### Example 18

### Controlled Release Combination Nimodipine and Cholinesterase Inhibitor*

Appropriate quantities of micronised nimodipine, cholinesterase inhibitor, fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| Cholinesterase Inhibitor | 20-40 |
| Fumaric Acid | 0-5 |
| Gelatine | 20-50 |
| Sorbitol | 0-10 |

* Cholinesterase Inhibitor may include Huperzine A, Tacrine, Donepezil, galanthamine or rivastigmine

### Example 19

### Controlled Release Combination Nimodipine and GABA-Analogue*

Appropriate quantities of micronised nimodipine, GABA analogue, fumaric acid, gelatine and sorbitol are added to water and heated to 80° C., continually stirring until in a homogeneous solution. The solution is then processed into solid minispheres at an appropriate, nozzle formation, flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| GABA-analogue | 20-40 |
| Fumaric Acid | 0-5 |
| Gelatine | 20-50 |
| Sorbitol | 0-10 |

* GABA analogue may include Piracetem or memenda

### Example 20

### Controlled Release Combination Nimodipine and Propentofylline

Appropriate quantities of micronised nimodipine, Propentofylline (Xanthine), fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| Propentofylline | 20-40 |
| Fumaric Acid | 0-5 |
| Gelatine | 20-50 |
| Sorbitol | 0-10 |

### Example 21

### Controlled Release Combination Nimodipine and Angiotensin II Receptor Inhibitor*

Appropriate quantities of micronised nimodipine, ATIIRI, fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| Angiotensin II Receptor Inhibitor | 20-40 |
| Fumaric Acid | 0-5 |
| Gelatine | 20-50 |
| Sorbitol | 0-10 |

* Angiotensin II Receptor Inhibitor include Losartan or Candesartan

### Example 22

### Controlled Release Combination Nimodipine and Anti-Coagulant

Appropriate quantities of micronised nimodipine, anti-coagulant, fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| Anti-coagulant | 20-40 |
| Fumaric Acid | 0-5 |
| Gelatine | 20-50 |
| Sorbitol | 0-10 |

### Example 23

### Controlled Release Combination Nimodipine and Nitric Oxide (NO) Donor*

Appropriate quantities of micronised nimodipine, NO donor, fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| NO-donor | 20-40 |
| Fumaric Acid | 0-5 |
| Gelatine | 20-50 |
| Sorbitol | 0-10 |

NO Donors include, but are not limited to Sodium 1-(Pyrrolidin-1-yl)diazen-1-ium-1,2-diolate, Disodium 1-[(2-Carboxylato)pyrrolidin-1-yl]diazen-1-ium-1,2-diolate, Sodium 1-(Piperazin-1-yl)diazen-1-ium-1,2-diolate, Sodium (Z)-1-(N,N-Diethylamino)diazen-1-ium-1,2-diolate, L-Arginine, Nitroglycerine, (Z)-1-[N-(2-aminoethyl)-N-(2-ammonioethyl) aminio]diazen-1-ium-1,2-diolate (DETA/NONOate) as well as NO-donor conjugated Nimodipine.

### Example 24

### Controlled Release Combination Nimodipine and Statin*

Appropriate quantities of micronised nimodipine, Statin, fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| Statin | 20-40 |
| Fumaric Acid | 0-5 |
| Gelatine | 20-50 |
| Sorbitol | 0-10 |

* Statins include Simvastatin, Atorvastatin, Pravastatin and Mevastatin

### Example 25

### Controlled Release Combination Nimodipine and α-Aminoamide*

Appropriate quantities of micronised nimodipine, α-aminoamide, fumaric acid, gelatine and sorbitol are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using an appropriate blend of Eudragit® RS, Eudragit® RL and fumaric acid to provide a 12-hour or a 24-hour release profile.

| | |
|---|---|
| Ingredients | % w/w |

Core Composition

| | |
|---|---|
| Nimodipine (Micronised) | 20-40 |
| Statin | 20-40 |
| Fumaric Acid | 0-5 |
| Gelatine | 20-50 |
| Sorbitol | 0-10 |

* α-aminoamide include NW-1029 (Newron)

### Example 26

### Combination Controlled Release Nimodipine/Yizhi

The core formulation was prepared as follows. Yizhi oil was prepared and heated to 65° C. The shell solution was prepared as follows: Appropriate quantities of gelatine, nimodipine and sorbitol were added to water and heated to 70° C. until in solution. The minicapsules were prepared using a Spherex Labo to produce 2-layer minicapsules, the core of which comprises Tacrolimus in an enhanced solubilised and permeabilised formulation. In addition, the core formulation does enable a degree of sustained release. The resulting minicapsules are further coating using Surelease® to provide a 12-hour or a 24-hour release profile.

| | |
|---|---|
| Ingredients | % w/w |

Core Composition

| | |
|---|---|
| Yizhi | 100 |

Shell Composition

| | |
|---|---|
| Gelatin | 40-50.0 |
| Nimodipine (micronised) | 30-40 |
| Sorbitol | 10.0 |

### Example 27

### Combination Controlled Release Nimodipine/Essential Oil*

The core formulation was prepared as follows. Essential oil was prepared and heated to 65° C. The shell solution was prepared as follows: Appropriate quantities of gelatine, nimodipine and sorbitol were added to water and heated to 70° C. until in solution. The minicapsules were prepared using a Spherex Labo to produce 2-layer minicapsules, the core of which comprises Tacrolimus in an enhanced solubilised and permeabilised formulation. In addition, the core formulation does enable a degree of sustained release. The resulting minicapsules are further coating using Surelease® to provide a 12-hour or a 24-hour release profile.

| | |
|---|---|
| Ingredients | % w/w |

Core Composition

| | |
|---|---|
| Essential Oil | 100 |

Shell Composition

| | |
|---|---|
| Gelatin | 40-50.0 |
| Nimodipine (micronised) | 30-40 |
| Sorbitol | 10.0 |

* Essential Oils may include, but are not limited to, EPA and DHA (different purity)

### Example 28

### Combination Controlled Release Nimodipine/Hydralazine

Hydralazine was added to a suitable solution of olive oil, Gelucire 44/01 and Labrafil 1944 heated and continually stirred until in solution. An appropriate amount of gelatine was heated and when in solution was homogenised with the hydralazine solution. The combined mix was passed through a vibrating nozzle to produce 1-layer minicapsules, the core of which comprises Hydralazine in an enhanced solubilised and permeabilised formulation. The resulting minicapsules may be further coated with a 23% weight gain of Eudragit RS30D to enable appropriate controlled release profile to maximise therapeutic benefits.

| | |
|---|---|
| Ingredients | % w/w |

Core Composition

| | |
|---|---|
| Hydralazine | 4.25 |
| Gelatin | 79.8 |
| Gelucire | 5.32 |
| Olive Oil | 6.11 |
| Labrafil MS 1944 | 4.52 |

The invention is not limited to the embodiments hereinbefore described which may be varied in detail.

1-17. (canceled)

18. A modified release dosage product comprising:
a plurality of minicapsules or minispheres containing a calcium channel blocker; and
a plurality of minicapsules or minispheres containing a calcineurin inhibitor.

19-73. (canceled)

74. A modified release dosage product as claimed in claim 18 wherein the minicapsules or minispheres containing the calcium channel blocker comprise a first population containing the calcium channel blocker for immediate release and a second population containing the calcium channel blocker for controlled release, and wherein the minicapsules or minispheres containing the calcineurin inhibitor comprise a first population containing the calcineurin inhibitor for immediate release and a second population containing the calcineurin inhibitor for controlled release.

75. A modified release dosage product as claimed in claim 18, wherein
the calcium channel blocker is nimodipine; and
the calcineurin inhibitor is tacrolimus.

76. A modified release dosage product as claimed in claim 74 wherein when exposed to a use environment substantially all of the nimodipine and substantially all of the tacrolimus are released within a 24 hour period.

77. A modified release dosage product as claimed in claim 74 wherein the minicapsules or minispheres containing nimodipine comprise a first population containing nimodipine for immediate release and a second population containing nimodipine for controlled release.

78. A modified release dosage product as claimed in claim 77 wherein the first population comprises minispheres containing nimodipine in a solid form for immediate release.

79. A modified release dosage product as claimed in claim 77 wherein the second population comprises minicapsules containing nimodipine, the capsule having a controlled release coating.

80. A modified release dosage product as claimed in claim 79 wherein the first population comprises minispheres containing nimodipine in a solid form for immediate release and wherein the second population comprises a first sub-population for release of nimodipine over a period of from 0 to 12 hours and a second sub-population for release of nimodipine over a period of from 12 to 24 hours.

81. A modified release dosage product as claimed in claim 18 wherein the minicapsules or minispheres containing tacrolimus comprise a first population containing tacrolimus for immediate release and a second population containing tacrolimus for controlled release.

82. A modified release dosage product as claimed in claim 81 wherein the first population comprises tacrolimus in a liquid form encapsulated within minicapsules.

83. A modified release dosage product as claimed in claim 81 wherein the second population comprises minicapsules containing tacrolimus, the capsule having a controlled release coating.

84. A modified release dosage product as claimed in claim 83 wherein the first population comprises tacrolimus in a liquid form encapsulated within minicapsules and wherein the second population comprises a sub-population for release of tacrolimus over a period of from 0 to 24 hours.

85. A modified release dosage product as claimed in claim 75 wherein, when exposed to a use environment, more than 40% of the nimodipine and more than 40% of the tacrolimus are released within 12 hours, and less than 15% of the tacrolimus and less than 15% of the nimodipine are released within 1 hour.

86. A modified release dosage product as claimed in claim 75 comprising:
(i) a hard gelatin capsule containing the nimodipine minicapsules or minispheres and the tacrolimus minicapsules or minispheres; or
(ii) a sachet containing the nimodipine minicapsules or minispheres and the tacrolimus minicapsules or minispheres; or
(iii) a pellet containing the nimodipine minicapsules or minispheres and the tacrolimus minicapsules or minispheres; or
(iv) a naso-gastric feeding product containing the nimodipine minicapsules or minispheres and the tacrolimus minicapsules or minispheres.

87. A modified release dosage product as claimed in claim 18 wherein the product contains high purity eicospentaenoic acid (EPA), or high purity docosahexaenoic acid (DHA), or a combination thereof.

88. A modified release dosage product as claimed in claim 18 wherein the product contains an acetylcholinesterase inhibitor.

89. A modified release dosage product as claimed in claim 18 wherein the product contains safinamide, or a dopamine analogue or agonist.

90. A modified release dosage product according to claim 75 comprising a plurality of minicapsules having a core which contains tacrolimus in a liquid, lipid-based formulation and an encapsulating material which contains micronized nimodipine.

91. A method for treating or preventing Parkinson's disease or Restless Leg Syndrome, comprising administering a therapeutically effective amount of the product of claim 89 to a subject in need thereof.

92. A method for treating or preventing a disorder, comprising administering a therapeutically effective amount of the product of claim 18 to a subject in need thereof, wherein the disorder is subarachnoid hemorrhage, stroke, transient cerebral ischemia, focal cerebral ischemia, Parkinson's disease, Restless Leg Syndrome, Alzheimer's disease, Amyotrophic Lateral Sclerosis (ALS), vascular dementia, or Huntington's disease.

93. A modified release dosage product as claimed in claim 18 comprising:
(i) a plurality of minicapsules or minispheres containing a hydroxylase inhibitor; or
(ii) a plurality of minicapsules or minispheres containing an anti-coagulant; or
(iii) a plurality of minicapsules or minispheres containing an angiotensin II receptor antagonist; or
(iv) a plurality of minicapsules or minispheres containing a nootrophic; or
(v) a plurality of minicapsules or minispheres containing a NMDA receptor antagonist; or
(vi) a plurality of minicapsules or minispheres containing a xanthine; or
(vii) a plurality of minicapsules or minispheres containing a cholinesterase inhibitor; or
(viii) a plurality of minicapsules or minispheres containing an opiate; or
(ix) a plurality of minicapsules or minispheres containing a migraine or cluster headache treatment or prophylactic; or
(x) a plurality of minicapsules or minispheres containing a depression treatment or prophylactic.
